(19) United States
(12) Patent Application Publication
(10) Pub. No.: US 2017/0333566 A1
(43) Pub. Date: Nov. 23, 2017

(54) Conjugated Proteins

(71) Applicant: AIMA Biotech, Franklin Park, NJ (US)

(72) Inventors: Seetharama A. Acharya, Cresskill, NJ (US); Vivek N. Acharya, Cresskill, NJ (US); Craig Andrew Branch, Cornwall, NY (US); Sangeetha Thangaswamy, Bronx, NY (US)

(21) Appl. No.: 15/526,575

(22) PCT Filed: Nov. 16, 2015
(86) PCT No.: PCT/US15/60876

§ 371 Date: May 12, 2017

Related U.S. Application Data
(60) Provisional application No. 62/079,805, filed on Nov. 14, 2014.

(51) Int. Cl. A61K 47/60; A61K 38/38; A61K 31/352; A61K 38/42; A61K 9/14

(52) U.S. Cl. CPC A61K 47/60; A61K 38/42; A61K 9/14; A61K 31/352; A61K 38/385

(57) ABSTRACT

Described herein are semisynthetic biopolymers comprising: a plurality of polyalkylene glycol chains, a protein, and an antioxidant; wherein the polyalkylene glycol chains are conjugated to the protein through a substituted succinimide linker. In some embodiments, the compounds described herein are conjugated proteins referred to as “semisynthetic supra perfusion agents”, “semisynthetic hybrid biopolymers”, “semisynthetic supra plasma expanders”, or the like. In some embodiments, these compounds mimic the same physiological consequences of high viscosity supra plasma expanders without being highly viscous—i.e. having a viscosity greater than blood.

### CROSS-REFERENCE TO RELATED APPLICATION

This application claims the benefit of priority from U.S. Provisional Application Ser. No. 62/079,805, entitled: “SUPRA PERFUSION AGENTS AND METHODS OF MAKING AND USING SAME”, filed Nov. 14, 2014, the contents of which are hereby incorporated herein by reference in their entirety.

### FIELD OF THE DISCLOSURE

The present application relates to the field of semisynthetic biopolymers that are useful for—inter alia—treating, preventing or ameliorating the symptoms associated with hematologic conditions and disorders.

### BACKGROUND

Supra perfusion occurs when colloidal plasma having a viscosity higher than blood is introduced into circulation. Supra perfusion, by high viscosity plasma expanders, is achieved by increasing intrinsic endothelial nitric oxide (NO) production through an increased shear thinning of red blood cells (RBC).

Since the grafting of polyethylene glycol (PEG) chains to albumin was discovered, conjugation of polymers to peptide and protein therapeutics to generate hybrid molecules has become a popular approach to alter and/or control their stability, biodistribution, pharmacokinetics and toxicology.

Sickle cell disease (SCD) was the first molecular disease to be characterized, and its pathology was considered as a consequence of the presence of a mutant Hb, HbS in the red blood cells (RBC) of these patients. The deoxy form of this mutant Hb polymerizes in vivo thereby clogging the arterioles and capillaries. However developing anti-sickling compounds as a therapeutic approach for the disease has been very elusive. Most of the early attempts towards the therapy have been to reduce the propensity of deoxy HbS to polymerize in vivo by attempting chemical modification of HbS in situ. Hydroxyurea therapy, which increases the level of fetal Hb in vivo thereby reducing the propensity of HbS to polymerize, is the most successful approach in this area so far.

However, the role of the increased rate of autoxidation in vivo to induce increased oxidative stress and to push the patient to a pro-inflammatory state and contribute to the disease pathophysiology, including enhanced ischemia reperfusion injury has not been appreciated until recently. Polymerization of HbS primarily clogs the arterial sides of the circulation, and contributes to hypoxia-reperfusion injury and hypoxia reperfusion mediated increased oxidative reactions contribute to the pro-inflammatory state of SCD. This leads to vaso-occlusion and clogging of the venial side of circulation. Thus SCD is primarily a molecular disease; but in recent years, has been described as a blood flow impairment disease, a pro-inflammatory disease, a pro-coagulant disease, and the like.

Despite all of the advances in this field, however, there remains a need for compounds that exhibit superior efficacy in increasing blood plasma volume and in increasing oxygen transfer from blood to surrounding tissue, which do not demonstrate vasoactivity. Embodiments of the present invention are designed to meet these needs.

### SUMMARY

In some embodiments, the compounds described herein are conjugated proteins referred to as “semisynthetic supra perfusion agents”, “semisynthetic hybrid biopolymers”, “semisynthetic supra plasma expanders”, or the like. In some embodiments, these compounds mimic the same physiological consequences of high viscosity supra plasma expanders without being highly viscous—i.e. having a viscosity greater than blood.

The present inventors have discovered that surface decoration of certain proteins, such as albumin (Alb) and hemoglobin (Hb), with multiple polyalkylene glycol chains, e.g. polyethylene glycol having a molecular weight of 3,000 or 5,000 Daltons (PEG 3K and 5K, respectively), introduces new properties of colloidal plasma expansion to these proteins. In some embodiments, these isoviscous semisynthetic hybrid biopolymers provide vasodilation, an increase in functional capillary density (FCD); and induce a state of supra perfusion without increasing cardiac work. This feature is unique to compounds of the present invention and distinguishes them from conventional colloidal plasma expanders. In some embodiments, compounds of the present invention are more efficient than the high viscosity supra plasma expanders in low wall shear environments, i.e. ischemic regions of low blood flow.

In some embodiments, the present invention provides a semisynthetic biopolymer comprising: a plurality of polyalkylene glycol chains, a protein, and an antioxidant; wherein the polyalkylene glycol chains are conjugated to the protein through a substituted succinimide linker.

Other embodiments provide a complex comprising: a plurality of polyalkylene glycol chains; a protein; and an antioxidant.

Still further embodiments provide a composition comprising: an antioxidant; a protein; and a plurality of polyalkylene glycol chains, wherein the polyalkylene glycol chains are conjugated to the protein.

In some embodiments, the present invention provides compounds that sense ischemic regions and attenuate ischemia related injuries. In some embodiments, compounds of the present are delivered in combination with an antioxidant. In some embodiments, compounds of the present invention provide a synergistic supra perfusion effect when administered in combination with an antioxidant.

As used herein, the phrase “in combination with an antioxidant”, includes, but is not limited to conjugation of an antioxidant to a semisynthetic supra plasma expander as described herein, or a physical mixture of the two.

In some embodiments, the PEGylation methods described herein involve functionalizing PEG with group specific reagents, so that the conjugation of PEG to the protein can be targeted to specific side chain groups of the proteins, such as amino, carboxyl, sulfhydryl or guanidino groups. In some embodiments, cysteine (Cys) residues can be introduced in a site specific fashion in place of preselected surface amino acid residues of proteins. In some embodiments, the thiol groups of the newly introduced Cys residues can be targeted for PEGylation using maleimide chemistry based PEG reagents. In further embodiments, serine (Ser) and/or threonine (Thr) residues are replaced with Cys residues, so that the net charge of the mutant protein is not altered as a result of the PEGylation.

Exemplary approaches to introducing thiols on the ε-amino groups of proteins as a means of increasing accessibility of the surface amino groups for PEGylation and targeting the PEG reagents to these sites by maleimide chemistry are described, for example, in U.S. Pat. No. 5,585,484.

In some embodiments, the present invention utilizes methods which are referred to as Extension Arm Facilitated PEGylation (EAF PEGylation), which has many advantages over conventional direct PEGylation techniques. In particular, EAF PEGylation reduces the direct interaction of the conjugated PEG-chains with the protein, without compromising any benefits. Without being bound by theory, the present inventors believe that enhanced molecular volume, high viscosity and high colloidal osmotic pressure, may play a role in neutralizing the vasoactivity of acellular Hb.

In some embodiments, the PEG-5K chains are conjugated to the surface amino groups using EAF PEGylation. In certain embodiments, the surface amino groups are first reacted with iminothiolane, which results in the extension of the side chain of lysine (Lys) residues by the linking of δ-mercapto butirimidyl chains, and the thiol groups of the extension arm are modified with maleimide PEG.

Some embodiments of the present invention provide a compound comprising a plurality of polyalkylene glycol chains conjugated to a protein such as albumin or ovalbumin, wherein the polyalkylene glycol chains are covalently bonded with a succinimide linkage at the intrinsic thiols from the Cys residues of the protein, and at other amino groups through an extension arm with a thiol at the distal end.

In some embodiments, the PEGylated proteins of the present invention are further complexed with (or conjugated to) an antioxidant. Some embodiments of the present invention provide compounds comprising a protein having a plurality of polyalkylene glycol chains and an antioxidant conjugated thereto.

It is yet another object of the invention to provide a method of increasing blood plasma volume using a compound comprised of a plurality of polyalkylene glycol chains conjugated to a protein such as Hb, albumin or ovalbumin.

It is yet another object of the invention to provide a method of increasing oxygen transfer from RBCs to the surrounding tissue using a semisynthetic hybrid biopolymer comprised of a plurality of polyalkylene glycol chains conjugated to a protein such as Hb, albumin or ovalbumin without significantly altering the oxygen carrying capacity of the system.

It is another object of the invention to provide a method of increasing blood plasma volume using a semisynthetic biopolymer comprised of a plurality of polyalkylene gycol chains, a protein, and an antioxidant.

It is yet another object of the invention to provide a method of increasing oxygen transfer from the blood to the surrounding tissue using a semisynthetic hybrid biopolymer comprised of a plurality of polyalkylene gycol chains, a protein, and an antioxidant.

### BRIEF DESCRIPTION OF THE DRAWINGS

FIG. 1 depicts a schematic representation of extension arm facilitated (EAF) conjugation of human serum albumin (HSA).

FIG. 2 depicts an experimental protocol for inducing vaso-occlusion in NY1DD.

FIG. 3 provides representative images of venules from transgenic sickle cell mice (NY1DD) treated with HSA-T12, PEG-Alb T12 and PEG-Alb in Hypoxia/reoyxgenation. (a) Wild type (b) NY mice (c) NY—untreated H/R (D) NY mice treated with HSA-T12 (E) NY mice treated with PEG-Alb-T12 (F) NY mice treated with PEG-Alb-T12, wherein the white arrow indicates the blood flow direction and the black arrow indicates leukocytes.

FIG. 4A provides experimental results showing NY1DD sickle mice receiving HSA-T12, PEG-Alb-T12, and PEG-Alb at the onset of reoxygenation and exhibiting marked reduction in leukocyte adhesion, with PEG-Alb-T12 having a normalizing effect.

FIG. 4B provides experimental results showing NY1DD mice receiving PEG-Alb and PEG-Alb-T12 at the onset of reoxygenation and exhibiting marked reduction in leukocyte emigration compared with untreated NY1DD mice, wherein the results show *P<0.03 vs. wild type; +P<0.00001 vs. normoxic NY1DD mice; and #P<0.0001 vs. untreated NY1DD mice subjected to a hypoxia reoxygenation protocol.

FIG. 5 shows experimental results for the wall shear rate and leukocyte adhesion of wild type and NY1DD mice in response to normoxia and hypoxia-reoxygenation with supra perfusion treatment and without treatment.

FIG. 6 shows experimental results for pretreatment with PEG-HSA-T12 (PEG-PNA), exhibiting improved arteriolar (A3) response to sodium nitroprusside (SNP) in sickle (S+S-Antilles) mice as compared to untreated sickle mice (P<0.003).

FIG. 7 shows exploded view of the structure of A) HSA-Tempo, B) EAF-HSA Tempo, and C) EAF PEG HSA-Tempo.

FIG. 8 shows the whole brain CBF % change/100 in wild type mice after saline or PEG-Alb.

FIG. 9 shows the % change in CBF/100 after PEG-Alb in wild type, Antilles, and BERK mice.

FIG. 10 shows CBF reactivity before and after PEG-Alb in wild type, S+S Antilles, and BERK mice.

FIG. 11 shows whole brain BOLD signal ratio after PEG-Alb in wild type, S+S Antilles, and BERK mice.

FIG. 12 shows evolving CBF during six minutes of hyperoxia in wild type and BERK mice.

FIG. 13 shows vascular contribution to the flow measurement in BERK mice.

FIG. 14 shows representative images showing postcapillary venules in the cremaster muscle microcirculation of Berk mice compared to QUE treated and wild type, wherein the black arrows indicate leukocytes and white arrows indicate the blood flow direction.

FIG. 15A shows a graph of leukocyte adhesion in QUE treated BERK mice at 50, 100, and 200 mg/kg doses compared to BERK and wild type mice.

FIG. 15B shows a graph of leukocyte emigration in QUE treated BERK mice at 50, 100, and 200 mg/kg doses compared to BERK and wild type mice.

FIG. 16A shows leukocyte adhesion in wild type and NY1DD mice in normoxia, hypoxia, and those treated with quercetin.

FIG. 16B shows leukocyte emigration in wild type and NY1DD mice in normoxia, hypoxia, and those treated with quercetin.

FIG. 16C shows red blood cell velocity in wild type and NY1DD mice in normoxia, hypoxia, and those treated with quercetin.

FIG. 16D shows leukocyte rolling flux in wild type and NY1DD mice in normoxia, hypoxia, and those treated with quercetin.

FIG. 16E shows wall shear rate in wild type and NY1DD mice in normoxia, hypoxia, and those treated with quercetin.

FIG. 16F shows volumetric flow rate in wild type and NY1DD mice in normoxia, hypoxia, and those treated with quercetin.

FIG. 17 shows in vivo blood flow in wild type and NY1DD mice in normoxia, hypoxia, and those treated with quercetin.

FIG. 18 shows in vivo blood flow in wild type, BERK, and BERK quercetin treated mice.

FIG. 19A shows red blood cell velocity in BERK untreated vs. BERK mice treated with quercetin.

FIG. 19B shows wall shear rate in BERK untreated vs. BERK mice treated with quercetin.

FIG. 20A shows leukocyte adhesion in wild type, BERK, and BERK quercetin treated mice.

FIG. 20B shows leukocyte emigration in wild type, BERK, and BERK quercetin treated mice.

FIG. 21 shows size exclusion chromatographic analysis of ovalbumin and PEG-ovalbumin.

FIG. 22 depicts the synthesis of a complex according to the present invention.

FIG. 23 depicts the impact of certain agents on functional capillary density in the standard-shock resuscitation protocol.

FIGS. 24A and 24B depict the impact that an exemplary compound of the present invention has on blood pressure and heart rate, respectively.

### DETAILED DESCRIPTION

In some embodiments, the present invention provides compounds comprising a plurality of polyalkylene glycol chains conjugated to a protein. In some embodiments the polyalkylene glycol is polyethylene glycol. In some embodiments, the protein is selected from: Hb, albumin and ovalbumin.

As used herein, “PEGlylated,” or “pegylation,” are related terms mean linking to polyalkylated glycol chains. As such, references to PEGylated albumin or PEGylated hemoglobin (PEG Alb or PEG Hb) refer to albumin or hemoglobin, that have been linked to one or more polyalkylated glycol chains. Analogously, PEGylated ovalbumin refers to ovalbumin that has been linked to one or more polyalkylated glycol chains.

In some embodiments, the present invention provides a semisynthetic biopolymer comprising: a plurality of polyalkylene glycol chains, a protein, and an antioxidant; wherein the polyalkylene glycol chains are conjugated to the protein through a substituted succinimide linker. In some embodiments, the polyalkylene glycol chains comprise polypropylene glycol (PPG) or polyethylene glycol (PEG). In some embodiments, the substituted succinimide linker comprises a thiol-containing moiety. In further embodiments, the substituted succinimide linker is a thiosuccinimidoethyl linker.

In some embodiments, at least one of the plurality of polyalkylene glycol chains comprises a polyethylene glycol having a molecular weight of from about 1,000 Daltons and about 30,000 Daltons. In some embodiments, at least one of the plurality of polyalkylene glycol chains comprises a polyethylene glycol having a molecular weight of from about 2,000 Daltons and about 10,000 Daltons. In some embodiments, at least one of the polyalkylene glycol chains comprises a polyethylene glycol having a molecular weight of from about 3,000 Daltons or about 5,000 Daltons. In some embodiments, the synthetic biopolymer comprises from about one to about eight polyalkylene glycol chains. In some embodiments, the synthetic biopolymer comprises six polyalkylene glycol chains.

In some embodiments, the protein is selected from hemoglobin, bovine derived hemoglobin, human derived albumin, bovine derived albumin, ovalbumin, and a combination of two or more thereof. In some embodiments, the protein comprises human derived albumin or ovalbumin. In some embodiments, the protein comprises hemoglobin. In some embodiments, the protein comprises intramolecularly crosslinked hemoglobin or intermolecularly crosslinked human derived albumin.

Some embodiments provide a method of increasing the volume of blood plasma in a subject in need thereof comprising administering to the subject an effective amount of a semisynthetic biopolymer as described herein. Other embodiments provide methods of increasing tissue oxygenation to the tissue of a subject in need thereof comprising administering to the subject, an effective amount of a semisynthetic biopolymer as described herein.

In some embodiments, the method comprises intravenous, interperitoneal or oral administration of the synthetic biopolymers of the present invention.

Some embodiments provide a complex comprising: a plurality of polyalkylene glycol chains; a protein; and an antioxidant. In some embodiments, at least one of said plurality of polyalkylene glycol chains is covalently bonded to said protein. In some embodiments, the covalent bonding of the polyalkylene gycol chain is performed using an extension arm facilitated (EAF) conjugation process.

In some embodiments, the antioxidant is conjugated to the protein in the complexes of the present invention. In some embodiments, the antioxidant comprises a flavonoid or a superoxide transmutase. In other embodiments, the antioxidant comprises a flavonoid selected from: 2-(3,4-dihydroxyphenyl)-3,5,7-trihydroxy-4H-1-benzopyran-4-one (quercetin) and isoquercetin. In some embodiments, the superoxide transmutase comprises 4-hydroxy-2,2,6,6-tetramethylpiperidin-1-oxyl (TEMPOL). In some embodiments, the quercetin comprises nanoparticle quercetin. In other embodiments, the quercetin is functionalized as a maleamide derivative.

In some embodiments, the present invention provides a method of increasing the volume of blood plasma in a subject in need thereof comprising administering to the subject an effective amount of a complex as described herein. In some embodiments, the present invention provides a method of increasing oxygen transfer from RBC into the tissue of a subject in need thereof comprising administering to the subject an effective amount of a complex as described herein.

Some embodiments provide a composition comprising: an antioxidant; a protein; and a plurality of polyalkylene glycol chains, wherein the polyalkylene glycol chains are conjugated to the protein.

Sickle cell disease (“SCD”) is a molecular disease as well as disease of blood flow impairment. In recent years, many therapies have emerged to improve the blood flow with the recognition that pathophysiology of SCD resembles many aspects of ischemia-reperfusion injury and situations where there is a decrease in the bioavailability of nitric oxide (NO) as well as the impairment of blood flow. The hemodynamic parameters are altered in transgenic mice due to the decrease in the bioavailability of NO, as result of increased oxidative stress, which eventually contributes to the loss of sensitivity for vasodilation in arteries. This vaso-occlusive event also further impairs blood flow on the venular side. Antioxidant therapies and anti-inflammatory therapies, together referred to as anti-adhesive therapies have shown the ability to attenuate the vaso-occlusive events accumulating on the venular side of circulation resulting in an improvement in the blood flow in transgenic sickle mice.

Unlike conventional colloidal plasma expanders, embodiments of the present invention induce vasodilation by increasing endothelial NO production; and increase functional capillary density and overall perfusion. Studies have shown that very large polymers with high viscosity (higher than blood) such as PEG, dextran, polylactic acid and starch are NO producing plasma expanders as they increase overall blood viscosity, wall shear and endothelial NO production. As a consequence, embodiments of the present invention improve microvascular flow and the efficacy of oxygen delivery to the tissues without an increase of oxygen carrying capacity. In some embodiments, PEG-Hb and PEG-Alb mimic the physiological impact of the high viscosity plasma expanders, despite having a viscosity equal to, or lower, than that of blood.

As used herein, “high viscosity” refers to a viscosity higher than the viscosity of blood under normal conditions.

In some embodiments, compounds of the present invention, induce their beneficial effects through a marginal increase in plasma viscosity.

As used herein, “P5” refers to a polyethylene glycol chain of about 5,000 Daltons; and “K6” refers to the linkage of six such chains to a protein, e.g. hemoglobin (Hb) or albumin. These two semisynthetic hybrid biopolymers are oxygen carrying and non-oxygen carrying low viscosity plasma expanders that exhibit active plasma expansion. In some embodiments, they improve both microcirculation and heart function at low plasma concentrations, without increasing cardiac work.

In some embodiments, the compounds of the present invention can be used to treat sickle cell disease (SCD), by attenuating the impairment of blood flow seen in the disease. It is believed that these beneficial effects are from their interaction with the endothelium on the arterial side of the circulation. In some embodiments, the anti-adhesive therapies of SCD exert their primary influence on the venial side of circulation. Accordingly, a combination of anti-adhesive therapy with the anticipated therapeutic effect of compounds of the present invention, sometimes referred to herein as “supra perfusion resuscitation fluids” or the like, afford an effective and novel combination therapy for SCD.

In some embodiments, the compounds of the present invention are prepared using an extension arm facilitated conjugation platform. In some embodiments, the present invention provides a biopolymer comprising a plurality of polyalkylene chains conjugated to a protein, which is further complexed or conjugated to an antioxidant. Some embodiments provide a polynitroxylated PEGylated Albumin with 12 copies of Tempo per mole (P5K6 Alb T12).

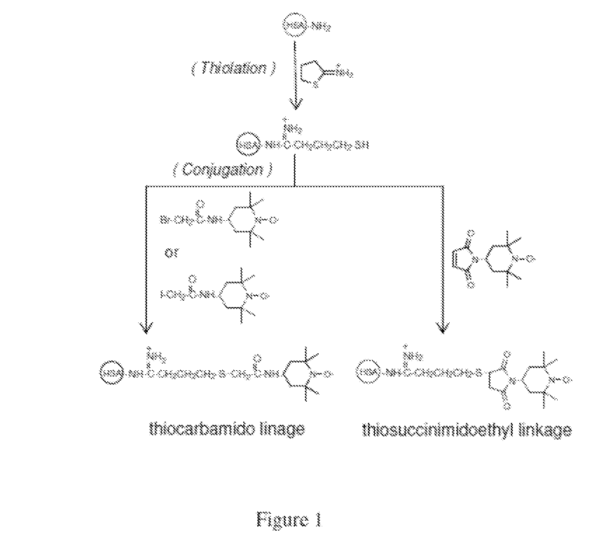

In some embodiments, the extension arm facilitated conjugation platform uses a click chemistry-like principle for conjugation of the antioxidant (e.g. tempol) to a protein. An exemplary method is shown in FIG. 1. In some embodiments, the method comprises the incubation of PEG-Alb with an antioxidant, or a derivative thereof, in the presence of iminothiol.

Attempts have been made to use gelatin as a plasma expander, after it has been EAF hexaPEGylated using maleimido phenyl urethane of PEG 5K. However, EAF hexaPEGylation reduced the efficacy of this molecule in a hemorrhagic shock model. Similarly, EAF PEGylation of dextran 70 amine with maleimide PEG 5K resulted in a product, EAF P5K5 Dextran 70, that was less efficacious than dextran 70. As demonstrated herein, the present inventors have discovered methods for overcoming these issues.

Ischemic reperfusion injury occurs when the blood supply to an area of tissue is cut off and the resupply oxygen is initiated. The incidence of ischemic injury is of common occurrence: myocardial infarction; stroke; and other thrombotic events, affect millions of individuals each year. In surgery, ischemic reperfusion injury occurs when the blood supply to an area of tissue is cut off

Some agents have been shown to improve functional capillary density in a transgenic sickle cell mouse under hypoxic, but the results have not been reproducible under normoxic conditions.

In some embodiments, compounds of the present invention increase the efficacy of antioxidant and or anti-inflammatory therapies. In some embodiments, compounds of the present invention may be suitable for use in treating, preventing or ameliorating the symptoms of thalassemia, trauma, traumatic brain injury, stroke, diabetes, cancer, organ transplant or neuro-degenerative diseases.

In some embodiments, the compounds of the present invention are referred to as semisynthetic hybrid biopolymers. In some embodiments, compounds of the present invention induce supra perfusion in vivo at viscosities significantly lower than that of blood, and in fact at viscosity levels that are lower than that of even conventional colloidal plasma expanders. In some embodiments, the compounds of the present invention induce vasodilation and a significant increase in functional capillary density. The vasodilator activity of the semisynthetic hybrid biopolymers of the present invention, stems from the fact that they increase the shear thinning of RBC resulting in improved levels of NO in the endothelium.

In some embodiments, the protein is a globular protein. In some embodiments, the globular protein is selected from hemoglobin, human (or bovine) serum albumin and ovalbumin. In EAF PEGylated proteins, a very large low packing density PEG shell is engineered around the central high packing density protein core with an intermediate zone of extension arms. This structure reduces the perturbation of the hydration layer of the protein by the PEG shell.

Extreme isovolemic hemodilution is an experimental animal model where systemic hematocrit of the subject is lowered below the transfusion trigger. This model therefore represents an experimentally induced anemia with concentration levels of Hb around 4 gm/dl, and provides a better picture of the ability of a compound to oxygenate a hypoxic region in the body. MP4, a comparative pegylated protein, was tested in this model, and was found to deliver little or no oxygen.

In some embodiments, compounds of the present invention prevent premature dumping of oxygen in the arteries, which can lead to vasoconstriction. In some embodiments, compounds of the present invention can deliver oxygen to regions where oxygen partial pressure is lower than the normal tissues i.e. the hypoxic areas of the body.

In some embodiments, the PEGylation is site-specific, e.g. at Cys-93((β).

In some embodiments, the compounds of the present invention carry a total PEG mass of ˜18K. Some embodiments have used the specificity of EAF hexaPEGylationof Hb, which is essentially dictated by the thiolation chemistry engineered using iminothiolane, and PEG chains longer than 2K (2,000 Daltons) and shorter than 5K (5,000 Daltons). In some embodiments, the approach introduces six copies of PEG 3K chains onto Hb. Some embodiments employ a 6 hr one-step EAF PEGylation platform, wherein the reaction is terminated by making the reaction mixture 10 mM with respect to N-ethyl maleimide.

In some embodiments, the compounds of the present invention have increased packing density of the PEG shell with a concomitant reduction in hydrodynamic volume.

In some embodiments, the structure of the PEG-shell of EAF P3K6 Hb counteracts the intrinsic hypertensive activity of Hb just as in EAF P5K6 Hb, but improves its ability to oxygenate the hypoxic regions as compared to EAF P5K6-Hb. In some embodiments, EAF P5K6-Alb provides a better functional capillary density (“FCD”) than EAF P5K6-Hb.

In some embodiments, compounds of the present invention increase delivery of oxygen to tissues without increasing the oxygen carrying capacity of blood. In some embodiments, compounds of the present invention demonstrate pseudo-plasticity, an intrinsic property which allows them to change their shape at low shear (or blood flow) and to increase the viscosity of the plasma in those regions. In some embodiments, this pseudo-plastic property facilitates shear thinning of RBC in the low shear region.

In some embodiments, compounds of the present invention are useful in reducing or attenuating ischemia reperfusion injury, and have application in diseases or conditions which cause ischemia reperfusion injury, e.g. SCD.

Ischemic reperfusion injury occurs when the blood supply to an area of tissue is cut off and the resupply oxygen is initiated. Ischemic injury commonly occurs, for example in myocardial infarction, stroke, and other thrombotic events, which affect millions of individuals annually.

Ischemic reperfusion injuries may also occur during surgery, when the blood supply to an area of tissue is cut off. This suggests that compounds or compositions that demonstrate pseudo-plasticity may have broader application than just in treating SCD.

Therapeutic strategies for ischemia reperfusion injuries have involved both antioxidant therapies and anti-inflammatory therapies, and in the case of SCD these are collectively referred to as anti-adhesive therapies.

In some embodiments, the present invention provides a semisynthetic biopolymer which demonstrates synergistic plasma expansion, when administered with an antioxidant. In some embodiments, the semisynthetic biopolymer comprises an extension arm facilitated pegylated protein, comprising a polyethylene glycol having a molecular weight of about 3,000 Daltons, wherein the protein is selected from hemoglobin (EAF P3K6 Hb), albumin (e.g., EAF P3K6 Alb) or ovalbumin (e.g., EAF P3K6 oAlb), and wherein the biopolymer comprises six copies of polyethylene glycol.

In some embodiments, the semisynthetic biopolymers of the present invention increase transfer of oxygen from RBC to hypoxic tissues, and/or provide a targeted increase in oxygenation of hypoxic tissues.

In some embodiments, the present invention provides conjugates comprising quercetin, which provide multi-functional therapeutic benefits (e.g. anti-oxidant, anti-inflammatory, anti-ischemic and chelation of divalent metal ions) that are effective in treating SCD.

In some embodiments, the present invention provides a physical mixture of a semisynthetic biopolymer of the present invention and a small molecular weight SCD therapeutic (e.g. quercetin) for treating, preventing or ameliorating a symptom of SCD.

Other embodiments provide a physical mixture of a supra plasma expander (e.g. a semisynthetic biopolymer according to the present invention) and quercetin conjugated albumin (semisynthetic enzyme mimetic) for treating, preventing or ameliorating a symptom of SCD.

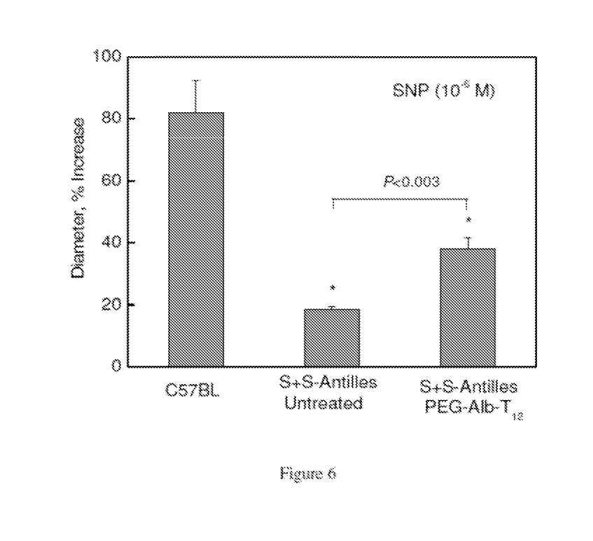

In some embodiments, the semisynthetic biopolymer of the present invention has a structure as shown FIGS. 7A, 7B or 7C, wherein HSA represents human serum albumin.

This invention will be described in greater detail by way of specific examples. The following examples are offered for illustrative purposes and are not intended to limit the invention in any manner. Those skilled in the art will readily recognize a variety of noncritical parameters which can be changed or modified to yield essentially the same results.

### EXAMPLES

### Example 1

### Materials and Methods

Human Serum Albumin (HSA) essentially fatty acids free, 2-imnothiolane (2-IT), 4-Maleimido Tempo (Mal-T), from Sigma. HbA is purified from the human erythrocyte lysate by DE-52 chromatography. Maleimidophenyl PEG5K (Mal PEG5K) is custom synthesized by BioAffinity, Inc., Rockford, Ill. Ingredients for PBS and other analytical chemicals are of HPLC grade from Sigma-Aldrich or Fisher Scientific, USA.

PEGylation of Human Serum Albumin (HSA): PEGylation of HSA is carried out by a thiolation-mediated maleimide chemistry-based protocol as detailed in Manjula et al., Conjugation of multiple copies of polyethylene glycol to hemoglobin facilitated through thiolation: influence on hemoglobin structure and function. Protein J. 2005; 24(3):133-463.

EAF Conjugation of Tempo (T12) to albumin and PEG Alb: 0.5 mM of PEG-albumin is incubated with 10 mM of 2-IT and 10 mM of 4-Maleimido Tempo in PBS (pH 7.4) at 4° C. for 16 hours. All chemical reagents in excess (unreacted) are removed using centricon [MWCO (molecular weight cut off) 50K from Millipore) spinning at 5000 rpm for 30 min. The washings are repeated three times. Alb-T12 is prepared using the same approach, but using Alb instead of PEG albumin.

Analytical Methods: SEC is performed on a Pharmacia FPLC system, at 25° C. using two HR10/30 Superose-12 columns connected in series with a 100 μL loop.

Intravital Microscopy: Intravital studies are approved by the Einstein Animal Care and Use Committee and were consistent with PHS recommendations. Male C57BL/6J control (n=4) and transgenic sickle (NY1DD) (n=12) mice weighing approximately 25-30 g (4-6 months old), are used. The mice are maintained on a standard diet and water ad libitum. Mice are anesthetized i.p. with 10% urethane and 2% a-chloralose in saline (5 ml/Kg). The animals are tracheostomised. In vivo microcirculatory observations are made in the open cremaster muscle preparation, and prepared according to the method of Baez S., An Open Cremaster Muscle Preparation for the Study of Blood Vessels by in vivo Microscopy. Microvas Res. 1973;5:384-94. Microscopic observations are carried out using a Nikon microscope (model E400; Morrell Instrument Company, Inc., Melville N.Y.) equipped with a Dage-MTI CCD television camera (model CCD-300T-RC, Dage-MTI Inc., Michigan City, Ind.) and a Sony U-matic video recorder (model V05800; Sony, Teaneck, N.J.).

Intravital measurements are initiated within 15 minutes of the surgical exteriorization of the tissue and completed within 30 minutes. Red cell velocity (Vrbc) and leukocyte adhesive behavior are determined in randomly chosen post capillary venules (diameter, ˜ μm). Vessel luminal diameter (D) is measured on-line using an image-shearing device (model 907, Instruments for Physiology and Medicine, San Diego, Calif.). Vrbc is measured along the vessel centerline using the ‘dual-slit’ photodiode and a velocity cross-correlator (model 102 BF, Instruments for Physiology and Medicine, San Diego, Calif.). The centerline Vrbc is converted to the mean Vrbc across the vessel diameter using a conversion factor of 1.6 (Vrbc/Vmean=1.6) as described. Volumetric flow rates (Q) are determined from Vmean and the vessel cross-sectional area (πD2/4) as described. Shear rates along the wall of microvessel of a given luminal diameter (D) are calculated using the relationship=8 Vmean/D.

Adherent leukocytes are counted along the length of a given venule, and expressed as average number of cells per 100 μm length of the vessel. A leukocyte is considered adherent if it remains stationary for longer than 30 sec. Trans-endothelial emigration (extravasation) of leukocytes is determined on-line as the number of interstitial leukocytes in the field of view adjacent (within 30 μm) to venules as described.

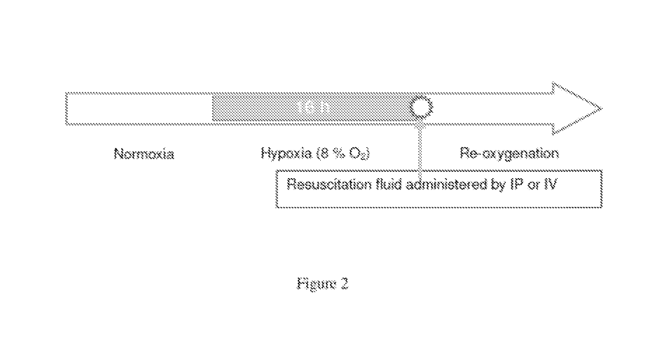

Transgenic sickle (NY1DD) mice are subjected to 18 hr of hypoxia (8% O2, 0.5% CO2, balance N2) followed by 3 hours of reoxygenation at ambient air (see, FIG. 2). NY1DD mice are selected based on their highly exaggerated response to hypoxia-reoxygenation compared with C57BL (control) mice. Microhemodynamic parameters (i.e., red cell velocity [Vrbc], wall shear rates and volumetric flow [Q]), leukocyte adhesion and leukocyte emigration are determined in the following groups of mice: 1) Normoxic wild type (C57BL) mice; 2) Normoxic sickle (NY1DD) mice; 3) NY1DD transgenic mice subjected to hypoxia-reoxygenation (untreated); 3) NY1DD mice subjected to hypoxia- reoxygenation and toploaded with PEG-Alb; 4) NY1DD mice subjected to hypoxia-reoxygenation and similarly toploaded with PEG-Alb-T12. In each group, mice are toploaded via tail vein with 4% of a given test substance at 10% of mouse blood volume. After 3 hours of reoxygenation, the cremaster muscle microvasculature is exposed and the tissue adjacent (within ˜30 μm) to postcapillary venules (the sites of inflammatory response) is examined for leukocyte adhesion and emigration as described.

The plasma expander like properties of EAF P5K6 Albumin conjugated with 12 copies of tempo Alb T12, EAF P5K6 Albumin and EAF P5K6 Hb are compared. The results are described in Table 1 (below).

As demonstrated by the data described in Table 1, dodeca-nitroxylation of albumin has very little influence on the plasma expander like properties of Alb. Similarly, dodeca-nitroxylation of P5K6-Alb has little influence on the plasma expander like properties of the molecule. Table 1 (below) describes the solution properties of certain protein-antioxidant conjugated products.

TABLE 1

| | Molecular Radius | COP | Viscosity |
|---|---|---|---|
| | (nm) | (mmHg) | (cP) |
| Alb-T12 | 4.0 | 10 | 1.0 |
| EAFP5K6-Alb-T12 | 7.9 | 44.8 | 2.3 |
| EAFP5K6-Alb | 7.8 | 44.7 | 2.3 |

### Example 2

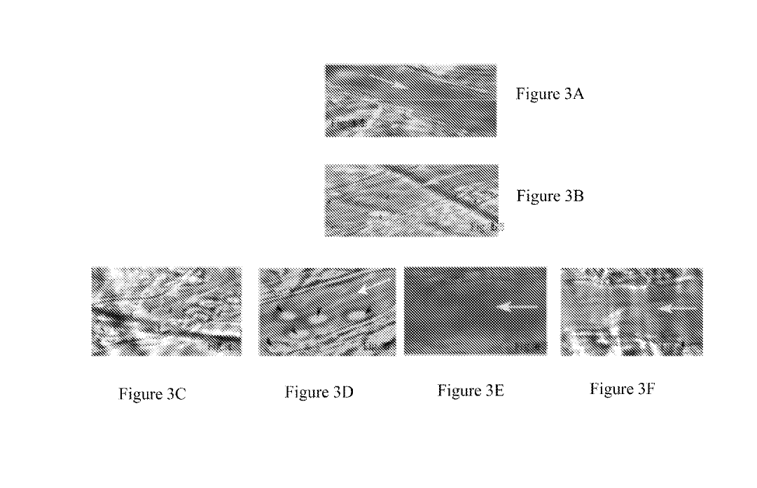

Intravital microscopic images of venules from transgenic sickle cell mice (NYIDD) subjected to hypoxia-reoxygenation and treated with therapeutic agents after hypoxia are illustrated in FIGS. 3A-3F. Images of the venule of the wild type mouse (C57 BL) show smooth endothelial walls (FIG. 3A). The NY1DD mice have a few leukocytes adhering to the endothelium of the venule (FIG. 3B). When NY1DD mice are subjected to hypoxia-reoxygenation without treatment with compounds of the present invention, circulation in the venule appears to be blocked (FIG. 3C), and a large amount of leukocytes (and deformed erythrocytes) adhere to the endothelial walls of the venules.

After treatment with Alb T12, leukocyte adhesion is slightly reduced (FIG. 3D). After treatment with P5K6-Alb-T12 (FIG. 3E), and P5K6-Alb (FIG. 3F); and before reoxygenation, clarity of the venules is essentially restored back to that observed in the venules of untreated sickle cell mice (FIG. 3C). These results demonstrate that the clearing of the venules in animals subjected to a hypoxia-reoxygenation protocol, is a function of PEGylation rather than T12. These results also suggest that compounds of the present invention would be effective in treating or preventing sickle cell disease, or symptoms associated therewith.

### Example 3

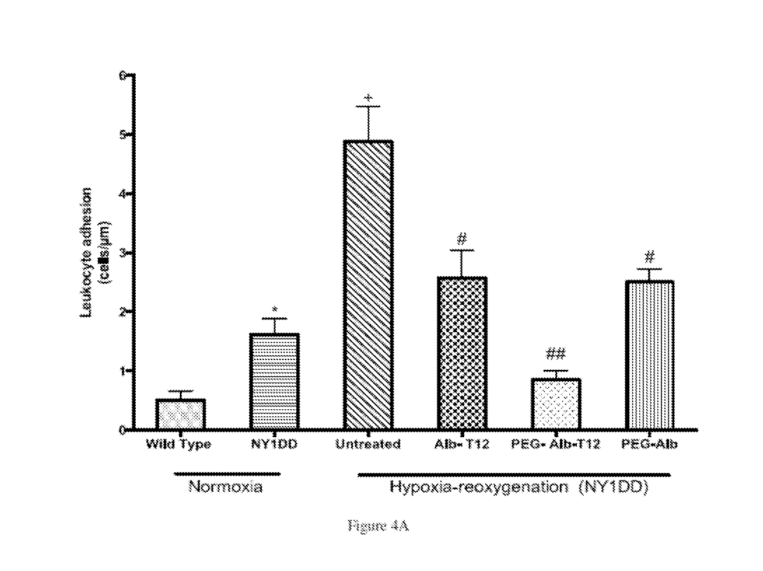

A quantitative analysis of inhibition of vaso-occulsion by an antioxidant, a PEGylated antioxidant (anti-oxidant conjugated supra perfusion fluids) and an exemplary PEGylated protein of the present invention—without antioxidant—is described in FIG. 4A. Even under normoxic conditions NY1DD mice show >2-fold increase in leukocyte adhesion in venules compared with wild type mice (P<0.04). The hypoxia-reoxygenation protocol in NY1DD mice caused a marked inflammatory response as evidenced by a ˜5-fold increase in leukocyte adhesion compared with normoxic NY1DD mice (P<0.00001). Infusion of Alb-T12 at the onset of reoxygenation achieves some noticeable reduction in leukocyte adhesion in comparison to untreated NY1DD. While treatment with P5K6-Alb-T12 after the onset of reoxygenation results in nearly a complete reduction in leukocyte adhesion as compared with untreated NY1DD mice (P<0.0001-0.00001).

Even under normoxic conditions, NY1DD mice exhibit an increased leukocyte emigration as compared to wild type mice (P<0.03, FIG. 4B). Hypoxia-reoxygenation in NY1DD mice results in a marked inflammatory response evidenced by 7.2-fold increase in leukocyte extravasation. Top loading P5K6-Alb-T12 after the onset of reoxygenation results in a pronounced reduction in emigrated leukocytes compared with untreated NY1DD mice subjected to hypoxia-reoxygenation (P<0.0001). PEG-Alb control also shows a significant improvement. In fact, leukocyte emigration in both treated groups was not significantly different from normoxic NY1DD mice.

NY1DD transgenic sickle mice have a normal hematocrit under normoxic conditions, but exhibit: i) an almost 60% decrease in Vrbc; and ii) a significant reduction in wall shear and blood flow (Q) as compared with wild type (C57BL) mice (see Table 3). Hypoxia-reoxygenation also causes a reduction in flow parameters. In contrast, all parameters showed a significant improvement when treated with P5K6-Alb-T12 at the onset of reoxygenation, such that the resulting values were significantly better than the base line values of NY1DD, and not significantly different from the baseline values in C57BL mice.

Significantly, the hemodynamic profile of a NY1DD mouse subjected to hypoxia-reoxygenation and treated with exemplary compounds of the present invention (with or without a conjugated anti-oxidant) is better than the pretreatment values. In fact, the flow and adhesion parameters are restored back to that of control C57 BL mouse (Table 3); whereas the hemodynamic profile of NY1DD treated with Alb-T12 after hypoxia returns only to that of the untreated NY1DD mouse. The hemodynamic profile of a NY1DD mouse treated with P5K6-Alb is comparable to the hemodynamic profile of a NY1DD mouse treated with P5K6-Alb-T12.

TABLE 2

| | Normoxia | | | | | |
|---|---|---|---|---|---|---|
| | Wild type | Sickle | Hypoxia-Reoxygenation (Sickle mice) | | | |
| Parameter | (C57BL mice) | mice | Untreated | HSA-T12 | PEG-Alb-T12 | PEG-Alb |
| Diameter (nm) | 28.7 ± 1.3* | 28.4 ± 1.5 | 28.4 ± 1.4 | 26.6 ± 0.91 | 28.6 ± 1.6 | 27.6 ± 1.4 |
| Red cell Velocity | 6.3 ± 0.6 | 2.5 ± 0.3+ | 1.7 ± 0.2** | 1.4 ± 0.22 | 6.1 ± 0.7# | 5.0 ± 0.7# |
| (mm/s) | | | | | | |
| Wall shear rate | 852.5 ± 102 | 472.4 ± 39+ | 211.5 ± 17** | 280.8 ± 45 | 819.8 ± 27.5# | 733.8 ± 23.7# |
| (s−1) | | | | | | |
| Volumetric flow | 2.9 ± 0.48 | 1.1 ± 0.19+ | 0.65 ± 0.08** | 0.51 ± 0.08 | 2.8 ± 0.64# | 1.7 ± 0.12# |
| (Q), nl/s | | | | | | |

Values are mean ± SE.
*Four to 6 venules were examined for hemodynamic parameters in each mouse (n = 3-4 each experimental group);
+P < 0.005-0.0001 vs. normoxic wild type controls;
**P < 0.05-0.023 vs. respective normoxic values for NY1DD mice;
#P < 0.004-0.00001 vs. untreated NY1DD mice subjected to hypoxia-reoxygenation

### Example 4

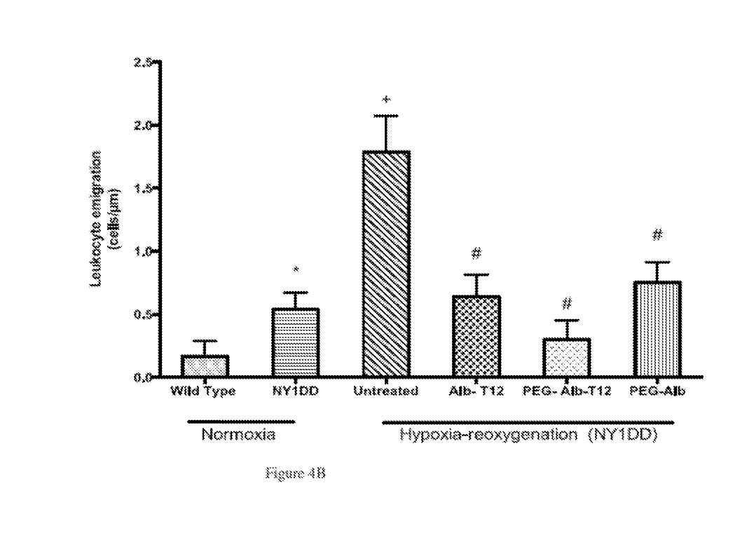

The attenuation of vaso-occlusion (see, FIG. 5) is used to demonstrate an improvement in the hemodynamic profile of NY1DD mice challenged with a hypoxia-reoxygenation protocol after the infusion of an exemplary compound of the present invention, at the onset of hypoxia. An improvement in wall shear correlates with an improvement in the hemodynamic profile and evidences the attenuation of adhesion, which is shown in FIG. 5. Alb-T12 has noticeable activity in reducing vaso-occlusion, as it establishes a wall shear that is comparable to the starting level before hypoxia. P5K6-Alb-T12 attenuates vaso-occlusion completely and restores wall shear values close to that of C57 BL, demonstrating a significant improvement over NY transgenic sickle mice that are not challenged with a hypoxia-reoxygenation protocol. Similar results are observed with PEG-Alb.

### Example 5

The ability of exemplary compounds of the present invention to improve the microvascular response to NO stimulated vasodilatation, as a consequence of the modulation of the oxidative stress, is evaluated. In particular, the response of cremaster arterioles (A3) to topical application of sodium nitroprusside (SNP, 10₋₆ M) after pretreatment with 150 μl of 4 g % P5K6-Alb-T12 is evaluated.

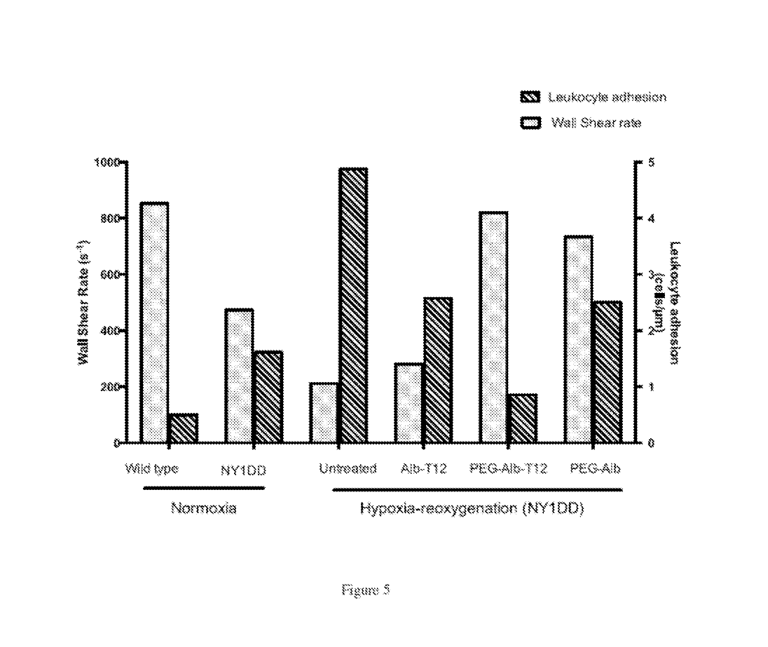

FIG. 6 describes the results of the vascular response to SNP in control C57BL and untreated S+S Antilles eighteen hours after T12 adduct treatment. Control C57BL mice exhibited approximately 80% increase in its arteriolar (A3) vascular diameter (P<0.01), while there is only an 18% increase in untreated transgenic SC mouse. In contrast, following PEG-Alb-T12 treatment, application of SNP induced a greater than two-fold increase in A3 diameters, exhibiting increased vascular sensitivity of NO in sickle mice on treatment with an exemplary compound of the present invention.

### Example 6

The ability of two exemplary compounds of the present invention and a comparative compound to attenuate vaso-occlusion in NY1DD transgenic sickle mice challenged with a hypoxia-reoxygenation protocol, is evaluated. While Alb-T12, P5K6-Alb-T12 and P5K6-Alb all attenuate vaso-occlusion in NY1DD transgenic sickle mice challenged with hypoxia-reoxygenation protocol, to some degree, the efficacy of the comparative compound (Alb-T12) is significantly lower than that of the exemplary compounds of the present invention (P5K6-Alb-T12 and P5K6-Alb). The exemplary compounds of the present invention, when transfused at 10% by top load after the hypoxia phase of the hypoxia-reoxygenation protocol, attenuate vaso-occlusion and normalize blood flow to a similar extent in this sickle mouse model, and both perform much better than Alb-T12.

In the hypoxia-reoxygenation challenged NY1DD, hemodynamics of the Alb-T12 treated mice are restored to a level comparable to that of the starting point, whereas the hemodynamics of the hypoxia-reoxygenation challenged NY1DD mice treated with P5K6-Alb-T12 are restored to the levels of the C57BL mice and are better than the pre-hypoxia levels. These results suggest that exemplary compounds of the present invention (e.g. P5K6-Alb-T12) can attenuate the damage present in NYDD mice before the hypoxia-reoxygenation protocol, and to return it to normal levels. Similar results are observed with P5K6-Alb. The reversal of the hemodynamics in NY1DD to a level of control C57BL suggests the therapeutic benefits supra perfusion to cumulative pathophysiological effects of sickle cell disease, including reduced systemic blood flow.

SCD therapeutic activity of supra perfusion resuscitation fluids are essentially from the improvement in blood flow, and attenuation of vaso-occlusion is presumably a consequence of improvement in blood flow. Besides a single dose of supra perfusion fluid also restores the sensitivity of the arterial endothelium in transgenic sickle mice S+S Antilles to some degree after a 16 hours treatment. Presumably this reflects a region-specific influence of the PEG Alb in hypoxic areas where blood flow and shear are reduced due to some blockage in the capillaries. It may be noted that EAF P5K6 Albumin is semisynthetic hybrid biopolymers of unique molecular structures. These have a central protein core of high packing density and an outer PEG shell of low packing density. The low packing density PEG shell is engineered onto the surface protein with a buffering zone of extension arms (FIG. 7). This endows the molecule with unique rheological properties, i.e. these semisynthetic supra perfusion resuscitation fluids are pseudo-plastic materials and increase their viscosity as a reverse correlate of wall shear. Besides these also increase functional capillary density, thus open up new connectivity between the arterial and venial system. PEG albumin, PEG Hb and other high viscosity plasma expanders that exhibit supra perfusion like dextran 500 and PEG 2M have been shown to increase the shear thinning of RBCs, this ability to increase the viscosity is a reverse correlate of shear. Besides, this activity of low viscosity plasma expander EAF P5K6-Alb is very distinct from other high viscosity supra perfusion resuscitation fluids. The EAF PEG-Alb and EAF PEG-Hb exhibits significantly higher shear thinning when mixed with blood at lower shear rates as compared to other high viscosity materials. Accordingly, it is conceivable that EAF P5K6-Alb can act as a molecular sensor of low blood flow (shear) in a region-specific fashion and change molecular shape to increase the viscosity and thus wall shear in these regions, leading to improved blood flow.

The studies with S+S Antilles establishes that a single dose of supra perfusion fluid achieves a significant reversal of the desensitization in the SCD mouse model.

### Example 7

Tissue oxygenation in extreme hemodilution models, as function of pattern of PEGylation, chemistry of PEGylation, and the influence of intramolecular crosslinking of Hb is investigated. A P5K2 version of αα-fumaryl Hb is generated, which exhibits a lower oxygen affinity than P5K2 Hb, but the pattern of PEGylation is same as that in P5K2 Hb. The oxygen affinity of P5K2-αα-fumaryl Hb is around 13 mm Hg as compared to about 8 to 9 mm of P5K2 Hb. This molecule did not improve the tissue oxygenation as compared to P5K2 Hb, it was around 6 mm Hg, if anything it was not as good as P5K2 Hb or P10K2 Hb. However, it was significantly better than MP4.

### Example 8

The plasma properties, total and plasma Hb concentrations, MAP and microvascular parameters of an exemplar compound of the present are compared with three comparative agents (Comp. Ex. I-III). Comp. Ex. I and II are hemoglobin derivatives, and Comp. Ex. III is dextran 70. Tables 3-5 describe data generated by the present inventors, as well as publicly available third-party data generated using similar evaluation techniques. The data described for Comp. Ex. I was reported by Winslow RM. MP4, A New Nonvasoactive Polyethylene Glycol-Hemoglobin Conjugate, Artif Organs. 2004; 28(9): 800-806. The data described for Comp. Ex. II was reported by Vandegriff K D, et al., Colloid Osmotic Properties of Modified Hemoglobins: Chemically Cross-linked Versus Polyethylene Glycol Surface-Conjugated, Biophys Chem., 1997; 69(1): 23-30. The data described for Comp. Ex. III was reported by Migita R, et al., Blood Volume and Cardiac Index in Rats After Exchange Transfusion with Hemoglobin-based Oxygen Carriers, J Appl Physiol. (1985). 1997; 82(6):1995-2002.

TABLE 3

| | Solution Properties | | | | |
|---|---|---|---|---|---|
| | Hb | Viscosity | COP | P₅₀ | |
| Composition | (g/dl) | (cP) | (mmHg) | (mmHg) | n |
| I | 6 | 2.1 | 89 | 9.5 | 2.2 |
| Comp. Ex. I | 4 | 2.2 | 49 | 5.4 | 1.2 |
| Comp. Ex. II | 8.4 | 5.2 | 167 | 5.4 | 1.2 |
| Comp. Ex. III | — | 2.8 | 50 | — | — |

TABLE 4

| Plasma Properties and Hb Concentration | | |
|---|---|---|
| | | Hb |
| Viscosity | COP | (g/dL) |

| Composition | (cP) | (mmHg) | Plasma | Blood |
|---|---|---|---|---|
| I | 1.4 | 23 | 1.5 | 4.7 |
| Comp. Ex. I | 1.4 | 23 | 1.1 | 4.8 |
| Comp. Ex. II | 1.5 | 28 | 1.6 | 5.1 |
| Comp. Ex. III | 1.4 | 17 | — | 3.7 |

TABLE 5

MAP and Microvascular Parameters

| | | | |
|---|---|---|---|
| | | Flow | Tissue |
| MAP | FCD | μcirc | pO₂ |

| | |
|---|---|
| Composition | Normalized to Baseline |

| | | | | |
|---|---|---|---|---|
| I | 0.79 | 0.64 | 0.80 | 7.6 |
| Comp. Ex. I | 0.87 | 0.68 | 0.62 | 1.8 |
| Comp. Ex. II | 0.9 | 0.71 | 0.55 | 4.1 |
| Comp. Ex. III | 0.65 | 0.38 | 0.8 | 1.1 |

[Hb]=hemoglobin concentration

Viscosity, measured at 37° C. and 150 s-1

COP=colloid osmotic pressure measured at 27° C.

P50=pO2 at which Hb is 50% saturated

n=cooperativity

MAP=mean arterial pressure

FCD=functional capillary density

Flow (μcirc)=mean arteriolar and venular blood flow.

¹Normalized to baseline, parameter divided by value at baseline and thus 1.0 represents no change, 0.5 and 1.5 represent a 50% reduction and 50% increase respectively

Mal-Propyl-PSK-Hb, MP4 and MP8 (Comp. Ex. I and II, respectively)

The major difference in the PEGylated protein with P5K6 and P3K6 protein is with respect to hydrodynamic volume; the PEGylation induced increase in the hydrodynamic volume is reduced by 60% as compared to that of EAF P5K6 Hb. But the PEG mass is only 40% lower than that in EAF P5K6 Hb. The PEG chains are packed more tightly in the P3K6 pattern, making the packing density of the PEG shell of this new material higher. A 6 gm % solution of this material, EAF P3K6 Hb has a viscosity comparable to that of a 4 gm % solution of EAF P5K6 Hb, i.e. these two represent is viscous solutions. The EAF P3K6 Hb, in extreme hemodilution model achieved an issue oxygenation level of nearly 8 mm Hg. slightly better than seen previously with P5K2 Hb as a 4 gm % solution. These results suggest that the structure of PEG shell of EAF P3K6 Hb is better than MP4 to serve as an adjunct to the red blood cells to increase its efficacy to oxygenate the tissues. The results described in Tables 3 to 5, illustrate that the packing density of the PEG shell in EAF P3K6 Hb and EAF P5K6 Hb are distinct, and this makes the release of oxygen from oxy-Hb in a closed system easier even though oxygen affinities are nearly comparable.

### Example 9

BERK, Antillies and WT (C57BL) mice are anesthetized with isoflurane and imaged in a 9.4 Tesla MRI (Agilent Direct Drive Spectrometer, Calif.). Measures are acquired prior to administration of PEG-albumin (PEG-Alb, 100 mg/kg IV in PBS, 180 ul via tail vein bolus over 120 seconds) and at 3 hours, 24 hours, 72 hours and 1 week following administration. Baseline imaging occurred at 3 or more days prior to PEG-Alb administration.

EMRI measures included brain perfusion (Arterial Spin Labeled MRI, using QUIPPS approach with vascular crushing), Diffusion Tensor Imaging (DTI, from which mean diffusivity was extracted), and BOLD imaging during a short period of inhalation with 100% O₂ from which an index of vascular responsiveness and de-oxy Hb change reflected in the BOLD signal intensity, was extracted, called the SO₂ parameter) which is related to the net change in T2 relaxation parameter. The effect of PEG-Alb on vascular reactivity is also assessed by administering a 3% CO₂ in Room Air challenge, during which the change in CBF was measured (CO₂/RA Perfusion Ratio, termed the reactivity measure (RM)).

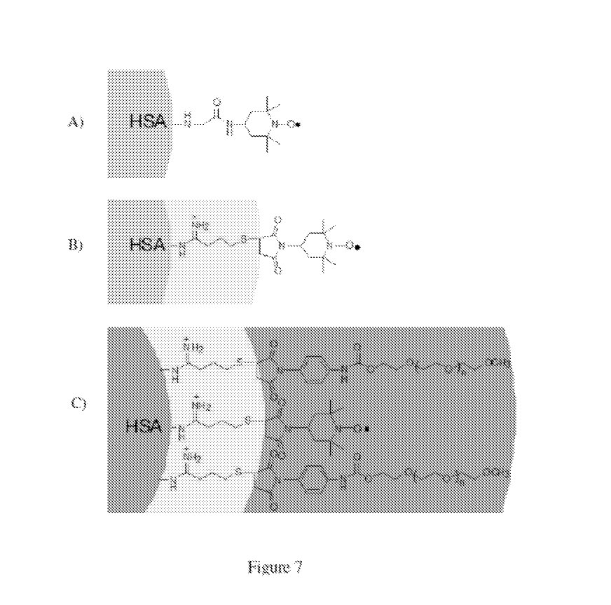

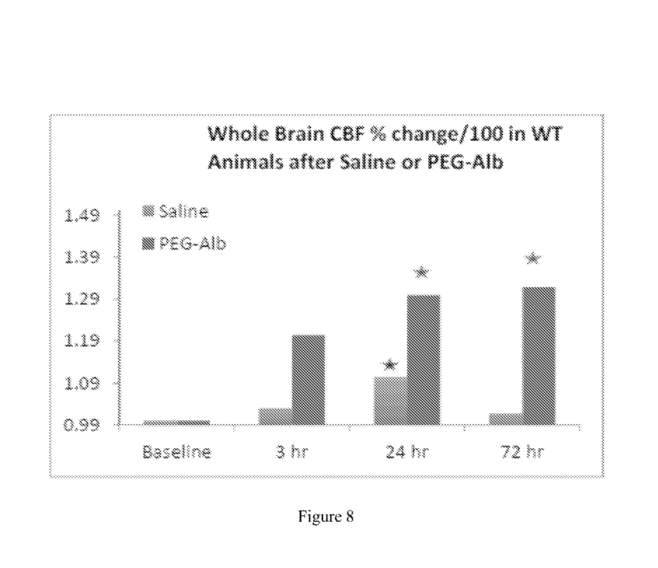

The effect of PEG-Alb administration in wild type (WT) Mice is shown in FIG. 8. As illustrated in FIG. 8, WT mice respond to PEG-Alb infusion with an increase in CBF which occurs both regionally (not shown) and in the whole brain blood flow. The percent change in CBF from baseline indicates that CBF increases and remained increased for up to 72 hours following PEG-Alb administration. The effect of an equivalent infusion of PBS is also shown (in blue).

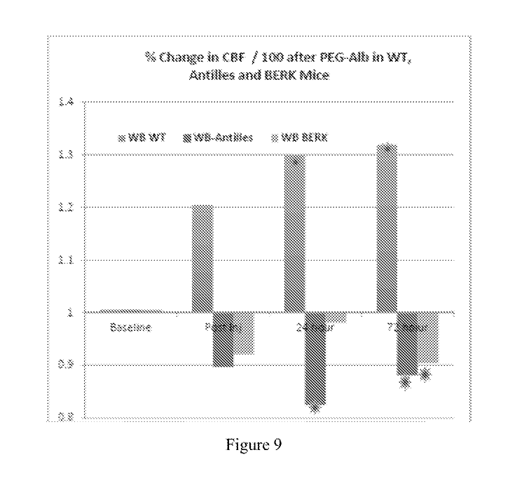

As shown in FIG. 9, SCD mice experience a decrease in CBF from their ‘hyperemic’ condition following administration of PEG-Alb, rather than an increase in CBF as was observed in the WT mice. The baseline CBF in SCD mice is severely hyperemic, which is consistent with observations of hyperemia observed in human SCD disease, and is thought to be due to antiregulatory increases in CBF compensating for anemic conditions. S+S Antiles mice are also hyperemic, but to a significantly lesser degree than was observed in BERK mice. Administration of PEG-Alb in WT mice also increases microvascular shear stress, increasing NO levels in the microvasculature, resulting in reduced vasogenic tone (increased arteriolar diameter and reduced vascular resistance) and increased CBF.

The improved wall shear, vasodilation and improved flow would be expected to reduce microvascular plugging from sickling cells, improving perfusion and resetting the CBF autoregulatory condition, allowing perfusion to return toward its normal state. However, the reduction in CBF that was observed in BERK and S+S Antiles suggests that they are not autoregulating, and that the increase in viscosity reduced velocity even with the associated increase in wall shear.

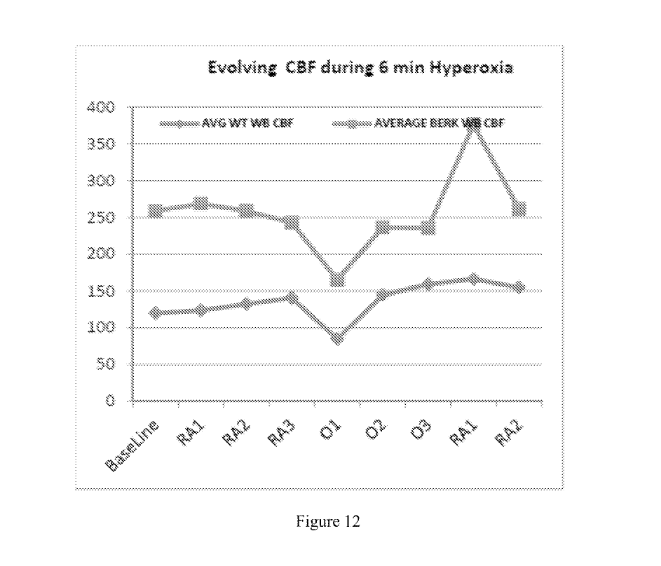

This does not mean that the PEG-Alb effect was negative—assessment of the reactivity of the cerebral vasculature to CO₂ inhalation before and after administration of the agent is illustrated in FIG. 10. Both BERK and S+S Antilles trended toward improved responsiveness to 3% CO₂ inhalation (FIG. 12) after PEG-Alb treatment, although only S+S Antilles exhibited a significant improvement at both 24 hours and 72 hours post-PEG-Alb administration. This improvement in vaso-responsiveness may be directly attributable to the decrease in CBF (suggesting that vasodilatory mechanisms may be exhausted in the transgenic SCD mice), or an improvement in NO regulation and sensitivity. The BERK study results demonstrate that the supra perfusion resuscitation fluids of the present invention attenuate the intrinsic vaso-occlusion and improve blood flow.

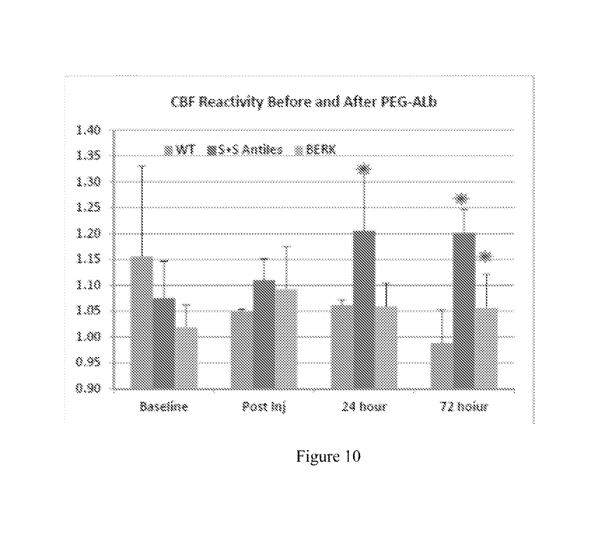

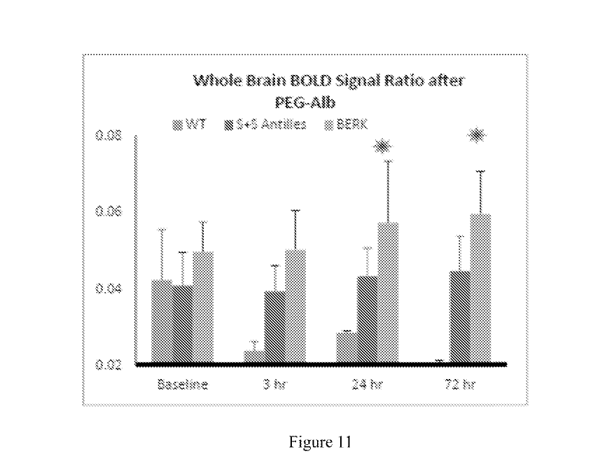

The autoregulatory system in these mice was explored in another way as well. Administration of 100% O₂ increases the partial pressure of oxygen in the blood plasma, although under normal conditions no change in Oxy-Hb saturation occurs. However, the increased O₂ in venous blood reduces the level of deoxy-Hb in venous blood, resulting in a reduction of the BOLD effect and consequent increase in BOLD signal intensity. The higher the deoxy-Hb level, the greater the change in signal intensity post-hyperoxia. WT Mice administered 100% O₂ exhibit an increase in BOLD signal intensity compared to BOLD signal intensity during inhalation of room air (i.e., a positive change in the Post-hyperoxia/Pre-hyperoixa signal ratio). Assuming that CBV remains unchanged, this effect can be directly attributed to a drop in deoxy-Hb/oxy-Hb ratio. Because both deoxy-Hb/oxy-Hb ratio and the cerebral venous blood volume effect the BOLD signal intensity, either CBV or CBF must be measured to properly interpret the BOLD signal change. It has been shown that CBF and CBV are related, and that oxygen delivery can be accurately assessed using the blood-oxygen-level-dependent (BOLD) MRI technique. BOLD images (which are T2-weighted MRI images whose image intensity are proportional to the level of deoxyHb in the tissues) are collected every 30 seconds while the animals breathe either room air (6 min), or 100% O₂ (6 min) and then returned to room air (4 min). The results of this experiment are expressed as the change in the ratio of the integrated signal intensity during the 100% O₂ inhalation period to the integrated signal intensity during the initial room air inhalation period. Return to initial baseline signal intensity is checked by comparing pre-hyperoxia to post- hyperoxia signal levels. FIG. 11 illustrates the effect of hyperoxia upon the BOLD signal in WT mice (blue n=2) administered PEG-Alb.

One interpretation of the findings are that hyperoxia induces a mild vasoconstriction, which results in a small change in cerebral blood volume, which is accentuated by administration of the PEG-Alb. The higher shear stress in the microvasculature may enhance the responsiveness of the microvasculature, inducing a slight reduction in CBV, and thus a reduced BOLD effect. The observed decrease in BOLD signal intensity after PEG-Alb administration (and hypothesis of increased vasoconstriction) would be consistent with the subtle decrease in CBF reactivity to CO₂ these animals experienced (FIG. 11) suggesting that cerebral blood volume changes associated with CO₂ vasodilation was attenuated by the PEG-Alb. However, this is in direct conflict with the increased CBF observed after administration of PEG-Alb. Alternatively, the increased CBF after Peg-Alb administration may have resulted in an uncoupling of CBF and oxygen metabolism, (i.e., overflow), which resulted in a decreased deoxy-Hb/oxy-Hb concentration, and thus a reduced BOLD Signal, decreasing the Post- Hyperoxia/Pre-Hyperoxia BOLD ratio. A reduced responsiveness to CO₂ would only accentuate this effect.

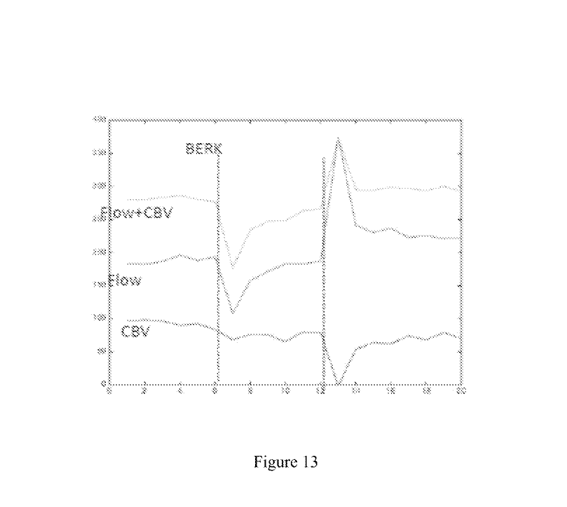

Finally, there is a third possibility. Hyperoxia (30% to 100%) is known to decrease blood pressure (BP), to decrease cardiac output (CO) and is thought to induce mild vasoconstriction. Thus, in the WT animals, hyperoxia would be expected to reduce cerebral perfusion pressure, and either cerebral arteriolar vasodilation would occur to maintain CBF, or CBF would drop. CBF does, in fact, drop transiently following induction of hyperoxia (FIG. 13), but quickly returns toward normal. It is likely that the since perfusion pressure drops transiently after induction of hyperoxia, the mild vasoconstriction that occurs would be quickly offset with a vasodilation, restoring CBF toward pre-hyperoxia levels. Administration of PEG-Alb would then attenuate this vasoconstriction/vasodilation because the improved flow caused by the induced supraperfusion would offset the transient change in perfusion pressure and the mild hyperoxia-induced vasoconstriction. This mechanism is consistent with the increase in CBF observed with PEG-Alb (FIG. 8).

BERK mice, by constrast, (n=4, green bars in FIG. 11) exhibited a trend toward increased BOLD signal after PEG-Alb treatment when hyperoxia was applied. Since CBF decreased in BERK animals after PEG-Alb administration, it is likely that the deoxy-Hb/Oxy-Hb ratio increased, consequently increasing the BOLD change ratio. If BERK autoregulatory mechanisms are dysfunctional, then it is likely that the supraperfusion agent has little acute effect on the Berk mouse, and thus hyperoxia, regardless of PEG-Alb administration is likely to reduce CBF and thus increase the BOLD Post-hyperoxia/Pre-hyperoxia ratio (which is usually observed). After PEG-Alb administration, our data suggest that the increased viscosity (which does not in this case induce vasodilatation) further reduces CBF, which results in a further increase in the BOLD post-hyperoxia/pre-hyperoxia ratio (or an increase in deoxy-Hb/Oxy-Hb ratio). FIG. 13 illustrated the changes that occur in BERK mice during hyperoxia, and illustrates both an enhanced drop in CBF after onset of hyperoxia, as well as a ‘rebound’ hyperemia after cessation of hyperoxia, probably in response to the transient increased oxygen debt. What is interesting is that the decrease in CBF and rebound may follow changes in BP and CO associated with the hyperoxia, suggesting a failure of myogenic autoregulatory mechansims. This would confirm the hypothesis that PEG-Alb will not improve BERK conditions acutely. However, even BERK mice exhibited an improved CO2 reactivity after PEG-Alb.

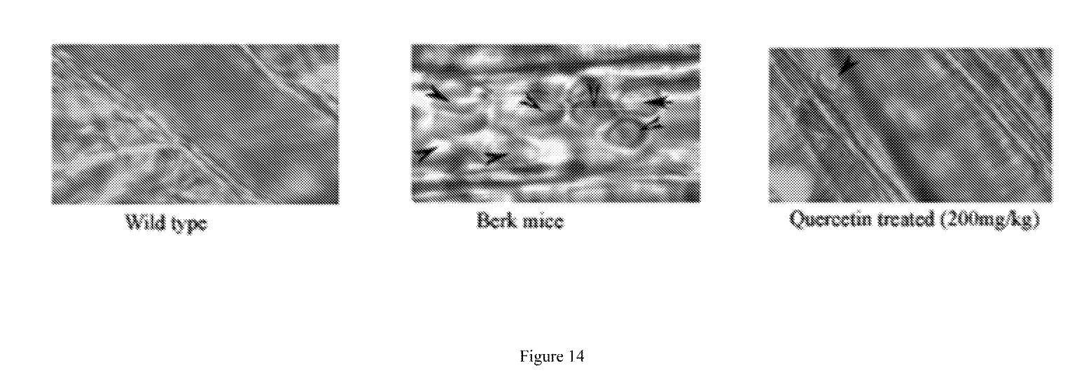

A further confirmation of these effects is evident in a measure of CBV obtained from an MRI experiment, in which the vascular contribution to the flow measurement is determined (FIG. 14) in BERK mouse. Importantly, this measurement is sensitive only to arteriolar blood volume (aCBV), not venous volume. As can be seen, the response to hyperoxia induction (red dotted line) resulted in a gradual drop in aCBV, which may indicate a sluggish arteriolar vasoconstriction, suggesting impaired autoregulatory systems. Thus, the drop in CBF was due to a drop in perfusion pressure. After resumption of room air (blue dotted line), a hyperemic response evolves. This hyperemia, however, may trigger an arteriolar vasoconstriction, and thus the sudden drop in CBV. Thus, while the autoregulatory system is impaired, it is not wholly absent.

Although CBF decreased in S+S Antilles mice after administration of PEG-Alb, their BOLD response was unremarkable for change. Since CBF decreased in S+S Antilles with PEG-Alb, one would expect an increase in the BOLD response (just as in the BERK mice). However, the vaso-reactivity in the S+S Antilles was enhanced (see FIG. 11). Since hyperoxia causes vasoconstriction and decreased cerebral perfusion pressure, it is possible that an enhanced vasoreactivity provided by the PEG-Alb facilitated a greater ability to compensate for the drop in perfusion pressure. Still, it remains to be clarified why these two animal models respond differently, but some insight may be attributable to the fact that BERK mice are anemic, while S+S Antiles are not, which results in a larger BOLD effect in BERK than other SCD mouse models, and may result is a greater degree of autoregulatory impairment.

### Example 10

EAF P3K6 Hb is evaluated for its ability to attenuate hypoxia-reoxygenation induced vaso-occlusion in NY1DD transgenic sickle mice. The viscosity of EAF P3K6 Hb is comparable to that of EAF P5K6 Hb, and has efficacy in clearing the veins.

Table 6 (below) describes the plasma expander properties and oxygen affinity, of two exemplary compounds of the present invention (II=EAF P3K6-Hb; III=EAF P5K6-Hb) versus a control (HbA). Oxygen affinity was evaluated in PBS at 37° C.

TABLE 6

| | Viscosity | | COP | | Oxygen Affinity | | Molecular |
|---|---|---|---|---|---|---|---|
| Com- | (cP) | | (mmHg) | | p50 | Hill | Radius |
| pound | 4% | 6% | 4% | 6% | (mmHg) | Number | (nm) |
| Control | 0.87 | 0.97 | 15.8 | 20.2 | 13.6 | 2.8 | 3.03 |
| II | 1.70 | 2.11 | 40.4 | 88.5 | 6.9 | 1.7 | 4.90 |
| III | 2.50 | — | 70.9 | — | 7.0 | 2.1 | 6.50 |

EAF P3K6 Hb also performs similarly to PEG-Alb and PEG-Alb-T12 in reducing the adhesion of leukocytes, but may not be as effective in attenuating emigration or rolling over of leukocytes; and restoring hemodynamics.

In extreme hemodilution models, differences between PEG-Hb and PEG-Alb are observed in terms of overall flow rate and FCD. In SCD mice treated with EAFP3K6-Hb, the bioavalability of NO is restored, but to a lesser extent than observed with EAFP5K6-Alb.

### Example 11

Berkeley (Berk) sickle transgenic mice which express exclusively human α- and (βS-globins with low levels of γ-globin (13-5%) generated by Paszty et al 1997. C57BL/6J are used as control wild type. A single dose of quercetin (“QUE”) at different concentrations (50, 100, 200 mg/kg body weight) intraperitoneally under normoxic conditions is administered. Three hours after QUE administration, in vivo intra-vital microscopic observation of post-capillary venules in cremaster muscle is performed. The luminal diameters of the venules (20-40 gm), centerline red blood cell velocity (Vrbc), adherent, emigrated and rolling leukocytes, and wall shear rates are measured.

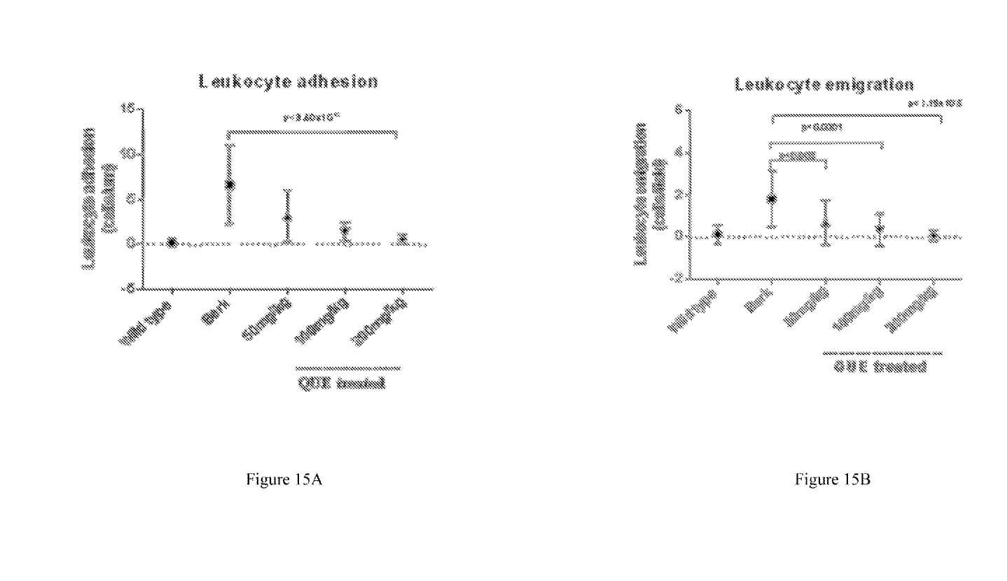

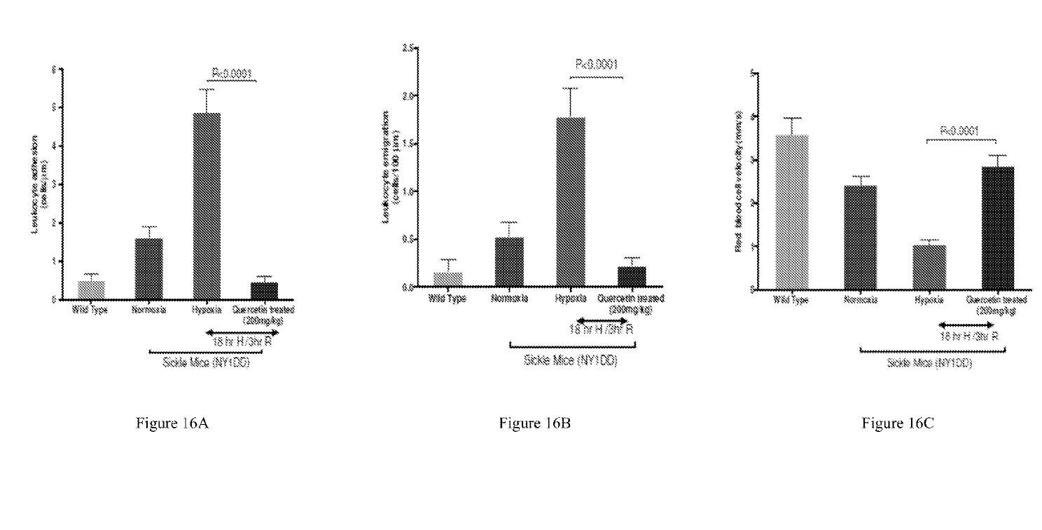

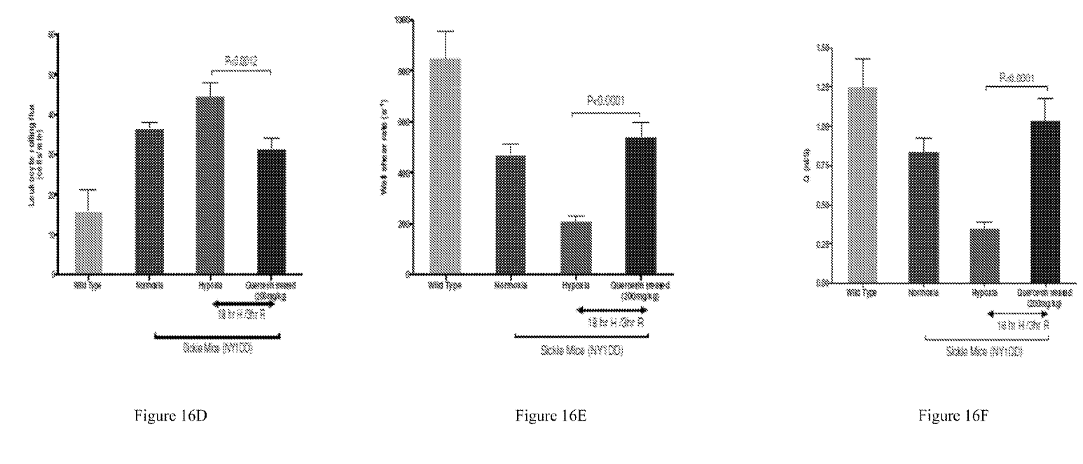

QUE treatment restored blood flow, as evidenced by complete disappearance of vaso-occlusion in the postcapillary venules of Berk mice (FIG. 15), with no significant difference in venular diameter compared to untreated Berk and wild type mice, at any of the dose levels tested (50, 100, 200 mg/kg). Howver, when compared to untreated Berk mice, a significant increase in the RBC velocity is demonstrated in a dose dependent fashion (treated: 1.74±1.3 mm/sec, 3.02±1.2 mm/sec, 3.4±0.90 mm/sec for 50, 100, 200 mg/kg dosing respectively vs. untreated 1.01±1.05mm/sec, p<0.05). A dose of 200 mg level completely neutralized the vaso-occlusion. Increases in wall shear rate (650.01±252.05 s⁻¹ vs. 180.12±165.02 s⁻¹, p<6.03×10⁻⁶) are also observed in QUE treated vs. untreated Berk. This improvement of blood flow in the postcapillary venules correlates well with observed decreases in leukocyte adhesion (FIG. 16A) and leukocyte emigration (FIG. 16B) in QUE treated Berk mice (for doses 50, 100, and 200 mg/kg) when compared to untreated Berk mice. Leukocyte rolling is also decreased for doses 100 and 200 mg/kg (p<0.007, p<0.0002 respectively) after treatment with QUE when compared to untreated Berk and wild type. These results provide support for the therapeutic application of flavonoids in SCD.

### Example 12

The effects of quercetin on hypoxia-reoxygenation injury in a transgenic sickle cell animal model are evaluated. Transgenic sickle cell mice (NY1DD) are used, in which β^{S}-globin forms symmetrical tetramers with human α-globin (42%) and with mouse α-globin (˜30%). In these mice, the total βS-globin levels are ˜75% βS of all β-globins.

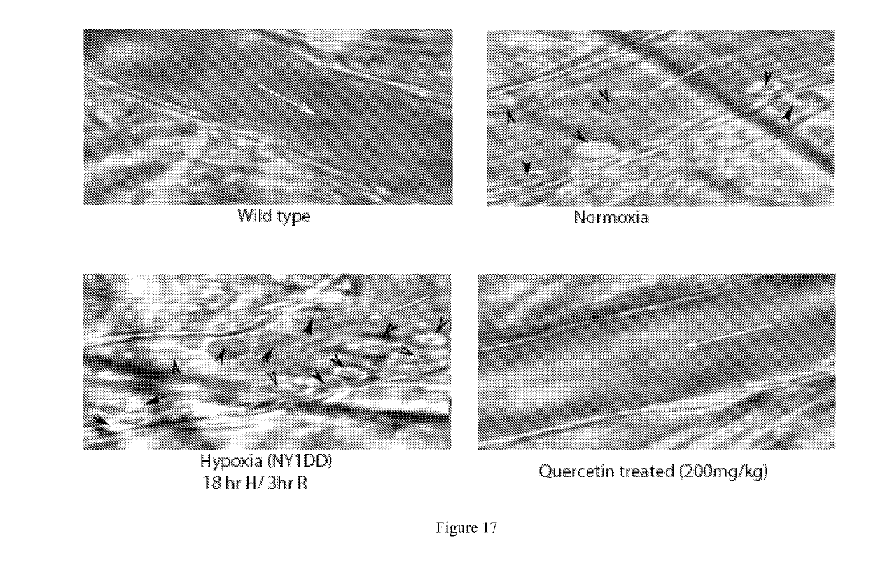

The experimental protocol consists of the following groups. Group 1: Normoxia wild type mice (C57BL/6J); Group 2: Normoxia NY1DD mice control; Group 3: NY1DD mice, subjected to 18 hours hypoxia (8% O2, 0.5% CO₂, balanced by N₂) followed by 3 hours reoxygenation at ambient air; and Group 4: NY1DD mice, subjected to 18 hours hypoxia (8% O₂, 0.5% CO₂, balanced by N₂) in Oxymax hypoxia chamber and immediately after 18 hours hypoxia, the animals are removed from the hypoxia chamber and querectin (200 mg/kg) is injected intra-peritoneally. After quercetin injection, the mice are kept for 3 hours in a reoxygenation environment at ambient air. Three hours after querectin administration, in vivo intra-vital microscopic observations of post-capillary venules in cremaster muscle are performed. The results of this experiment are shown in FIG. 17A through 17F.

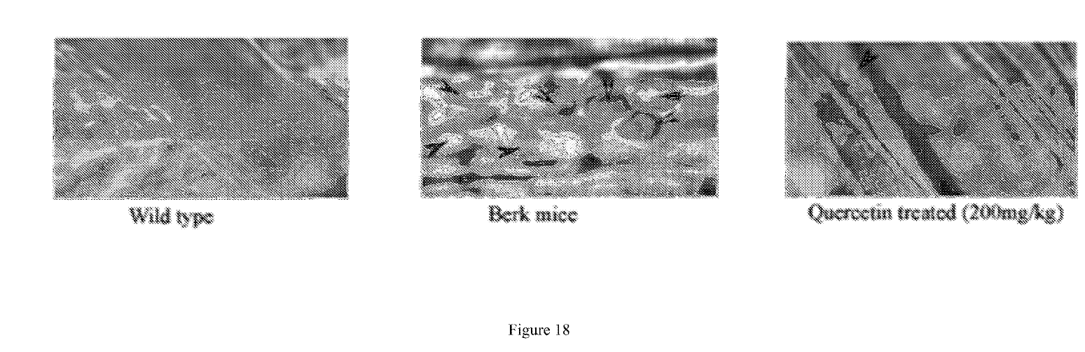

FIGS. 17A through 17F demonstrate that the quercetin treated Group 4 mice exhibited favorable therapeutic effects. FIG. 17A shows that Group 4 mice exhibited a reduction in leukocyte adhesion similar to wild type mice, rather than the increased adhesion of Group 2 and Group 3 mice under either normoxic or hypoxic conditions, respectively. The quercetin treated mice of Group 4 also exhibited reduced leukocyte emigration, similar to wild type (FIG. 17B). The Group 4 mice analogously exhibited a favorable increase in red blood cell velocity (FIG. 17C), leukocyte rolling flux (FIG. 17D), wall shear rate (FIG. 17E), and in volumetric flow rate (FIG. 17F). Furthermore, as demonstrated in FIG. 18, quercetin treatment restored blood flow, as evidenced by complete disappearance of vaso-occlusion in the postcapillary venules after the hypoxia-reoxygenation protocol. These results support a therapeutic application for flavonoids in SCD.

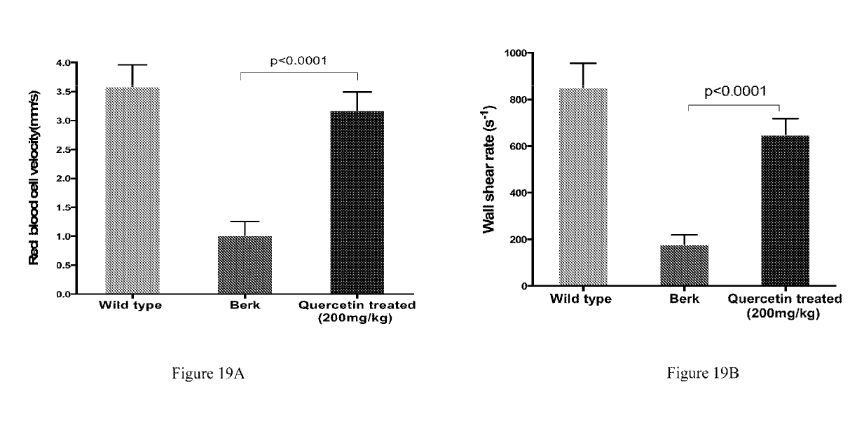

Berkeley (BERK) sickle transgenic mice which express exclusively human α- and β^{S}-globins with low levels of y-globin (γ3-5%) also confirm the protective effect of quercetin in vivo. C57BL/6J were used as control wild type. A single dose of quercetin is injected at different concentrations (50, 100, 200mg/kg body weight) intraperitoneally under normoxic conditions. Three hours after quercetin administration, in vivo intra-vital microscopic observation of post-capillary venules in cremaster muscle is performed (see, FIG. 19).

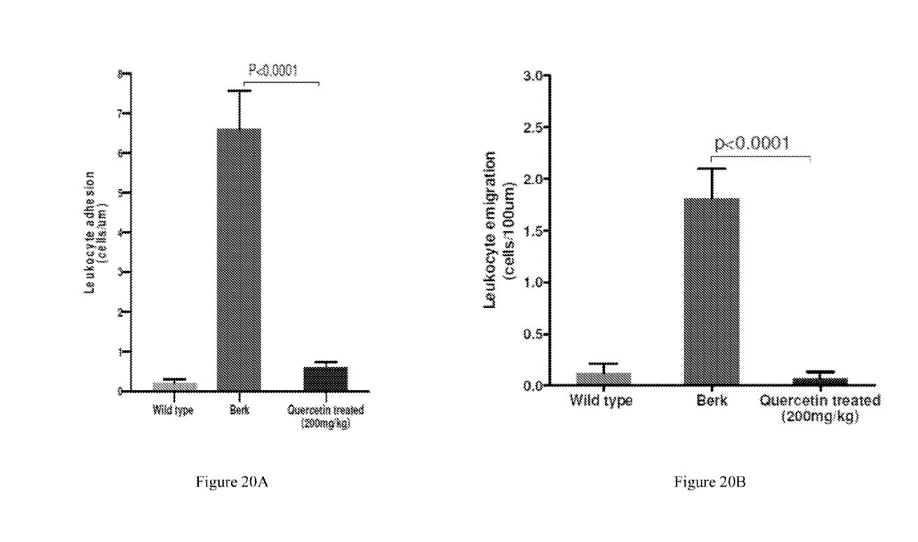

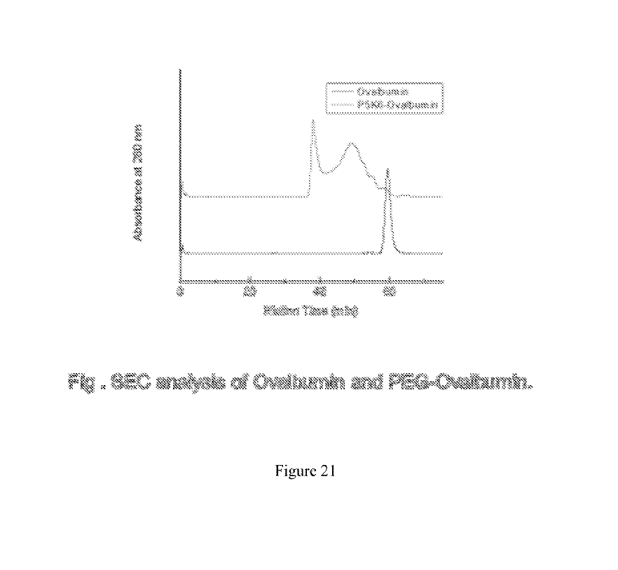

While no significant differences in venular diameter were noted with quercetin treatment (200 mg/kg) compared to untreated Berk and wild type mice (FIG. 19), a significant increase in the RBC velocity was demonstrated at 3.4±0.90 mm/sec vs. untreated 1.01±1.05 mm/sec, (p<0.05) (see, FIG. 20A). Increased wall shear rate (650.01±252.05 s⁻¹ vs. 180.12±165.02 s⁻¹, p<6.03×10-6) is also observed in quercetin treated vs. untreated Berk, as shown in FIG. 20B. This improvement of blood flow in the postcapillary venules correlates well with observed decreases in leukocyte adhesion (FIG. 21A) and leukocyte emigration (FIG. 21B) in quercetin treated Berk mice (200 mg/kg) when compared to untreated Berk mice. Leukocyte rolling is also decreased at dose 200 mg/kg after treatment with quercetin when compared to untreated Berk and wild type.

### Example 13

Quercetin can be functionalized as a malemide derivative by reacting the hydroxyl groups with PMPI (para maleimido phenyl isothiocyanate), to produce a macromolecular antioxidant Alb- Quer12 adduct as well as EAF P5K6-Alb-Quer12. The conjugated quercetin product will have an increased half-life in circulation; and thus, a lower dose can likely be used to achieve the desired effect.

### Example 14

### EAF PEGylation of Ovalbumin

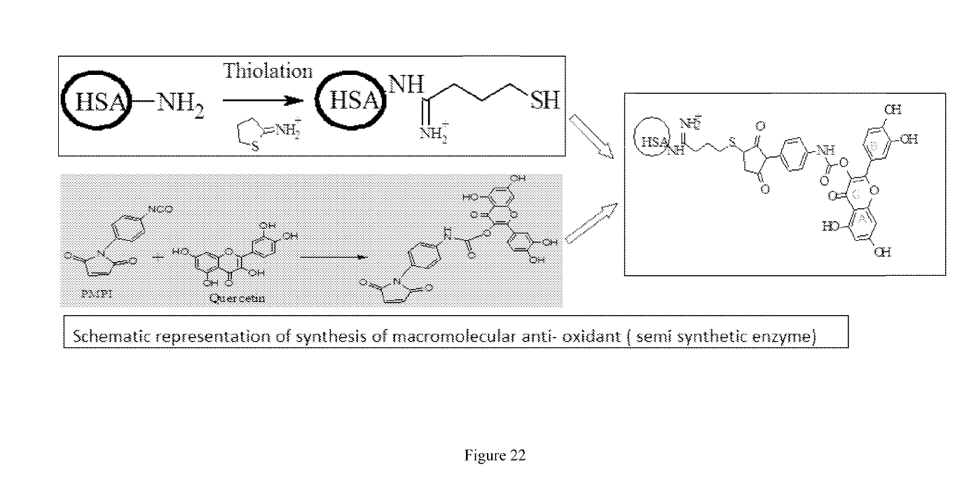

0.5 mM of ovalbumin, 10 fold molar excess of 2-imiothiolane and 20 fold molar excess of maleimideo phenyl urethane of PEG 5K are incubated overnight at 4° C. The size exclusion chromatographic analysis of the product shows that almost all of the ovalbumin has been PEGylated (FIG. 22). The elution pattern is comparable to that of EAF hexaPEGylayed albumin, but for a very small portion of the PEGylated material elutes at the void volume of the column, reflecting very high hydrodynamic volume for this material.

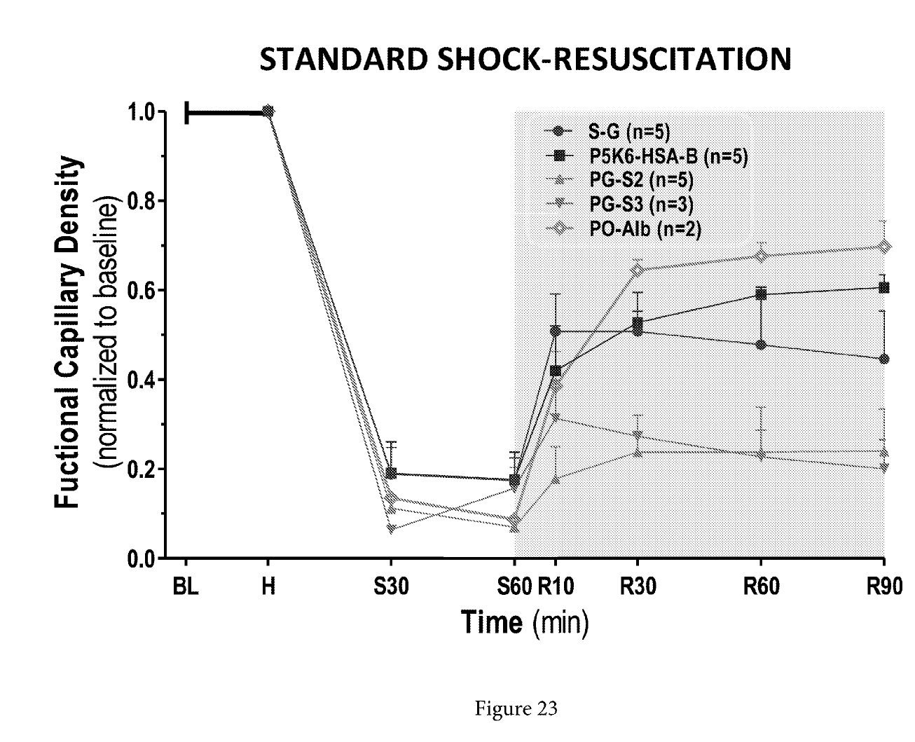

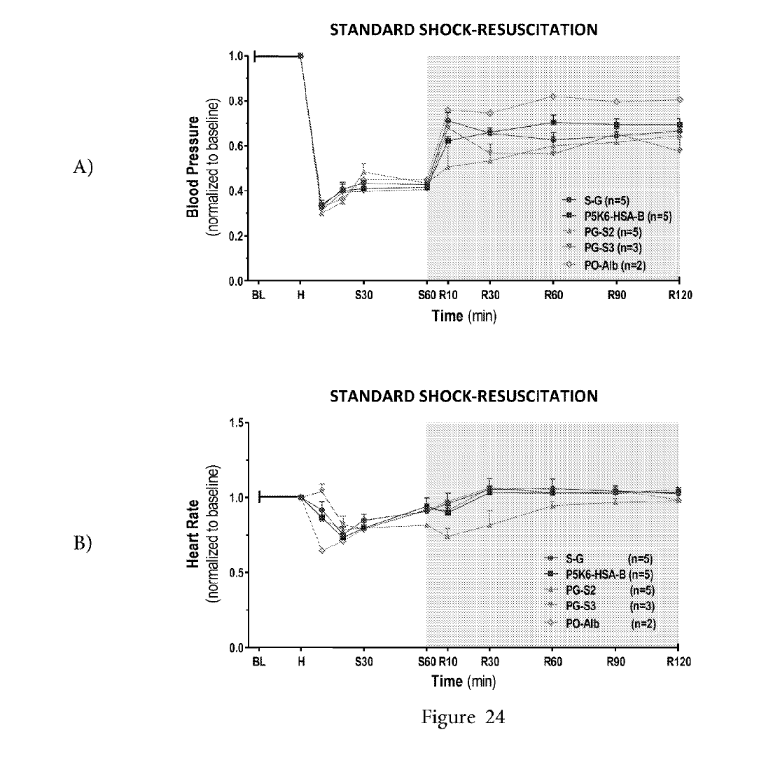

Reconstitution of P5K6-ovalbumin as a 4 gm % solution shows efficacy in a hamster model for hemorrhagic shock. FIGS. 23 and 24 (A & B) describe the impact that an exemplary pegylated ovalbumin according to certain embodiments of the present invention has on recovery of functional capillary density after hemorrhagic shock; and its impact on blood pressure and heart rate, respectively. Table 7 (below) describes data obtained from blood samples after a standard-shock resuscitation protocol.

TABLE 7

| | COP | Viscosity |
|---|---|---|
| | (mm Hg) | (cP) |
| IV | 14.2 ± 0.2 | 1.29 ± 0.06 |
| Comp. Ex. IV | 12.1 ± 0.4 | 1.04 ± 0.04 |
| Comp. Ex. V | 13.1 ± 0.2 | 1.02 ± 0.07 |
| Comp. Ex. VI | 11.1 ± 0.5 | 1.09 ± 0.09 |
| Comp. Ex. VII | 12.0 ± 0.5 | 1.03 ± 0.02 |

### Example 15

A new version of PxK6 Hb, (Propyl PEG 2K)6-Hb and (Propyl PEG2K)6-αα-Hb, is generated. In these molecules, Cys-93((β) is free where it is the only site PEGylated in P5K2-Hb and P5K2 αα-Hb. The oxygen affinity of these compounds are 6.5 mm Hg and 14.5 mm Hg respectively. These PEG-Hbs carry a total PEG mass of 12K conjugated to Hb as six copies of a 2K PEG chain. Tissue oxygenation with these molecules was 4.2 mm Hg and 5.4 mm respectively, with very limited influence on the overall micro-circulatory properties. This value is still lower than the value for P5K2-Hb in this system (7.0 mm Hg). These results confirm that the pattern of the PEGylation, as well as the amount of PEG conjugated, dictates the structure of PEG shell which impacts tissue oxygenation much more than the P50 of the PEGylated Hb.

It is intended that any patents, patent application or printed publications, including books, mentioned in this patent document be hereby incorporated by reference in their entirety.

As those skilled in the art will appreciate, numerous changes and modifications may be made to the embodiments described herein, without departing from the spirit of the invention. It is intended that all such variations fall within the scope of the claimed invention.

1-39. (canceled).

40. A complex comprising:
a plurality of polyethylene glycol chains,
a protein, and
an antioxidant;
wherein the polyalkylene glycol chains are conjugated to the protein through a thiosuccinimidoethyl linker; and
wherein the polyethylene glycol has a molecular weight of about 3,000 Daltons.

41. The complex according to claim 40, comprising six polyalkylene glycol chains.

42. The complex according to claim 40, wherein the protein is selected from hemoglobin, human derived albumin, bovine derived hemoglobin, bovine derived albumin, ovalbumin, intramolecularly crosslinked Hb, intermolecularly crosslinked human derived albumin, and a combination of two or more thereof.

43. The complex according to claim 42, wherein the protein comprises human derived albumin or ovalbumin.

44. The complex according to claim 42, wherein the protein comprises hemoglobin.

45. The complex according to claim 42, wherein the protein comprises intramolecularly crosslinked Hb or intermolecularly crosslinked human derived albumin.

46. The complex according to claim 40, wherein the antioxidant comprises a flavonoid or a superoxide transmutase.

47. The complex according to claim 46, wherein the antioxidant comprises a flavonoid selected from: 2-(3,4-dihydroxyphenyl)-3,5,7-trihydroxy-4H-1-benzopyran-4-one (quercetin) and isoquercetin.

48. The complex according to claim 47, wherein the quercetin, comprises nanoparticle quercetin.

49. The complex according to claim 48, wherein the quercetin is functionalized as a maleamide derivative.

50. A method of increasing the volume of blood plasma in a subject in need thereof comprising administering to the subject an effective amount of a complex according to claim 40.

51. A method of increasing oxygen transfer from RBC into the tissue of a subject in need thereof comprising administering to the subject an effective amount of a complex according to claim 40.

52. A method of treating, preventing or ameliorating a symptom of sickle cell disease, comprising administering a complex according to claim 40, to a subject in need thereof.

53. A method of treating, preventing or ameliorating a symptom of sickle cell disease, comprising administering a compound comprising a protein conjugated to a plurality of polyalkylene glycol chains; and an antioxidant to a subject in need thereof.

54. The method of claim 53, wherein the compound and the antioxidant are administered concurrently.

55. The method of claim 53, wherein the compound and the antioxidant are sequentially administered.

56. The method of claim 53, wherein the compound and the antioxidant are contained within a single composition.

57. The method according to claim 56, wherein the composition further comprises a pharmaceutically acceptable carrier.
